(19) United States
(12) Patent Application Publication
(10) Pub. No.: US 2025/0346864 A1
(43) Pub. Date: Nov. 13, 2025

(54) MODULATION OF SATELLITE CELL POLARITY AND ASYMMETRIC CELL DIVISION

(71) Applicant: Ottawa Hospital Research Institute, Ottawa (CA)

(72) Inventors: Michael A. RUDNICKI, Ottawa (CA); Kasun KODIPPILI, Ottawa (CA)

(21) Appl. No.: 18/868,700

(22) PCT Filed: May 24, 2023
(86) PCT No.: PCT/US2023/067421

§ 371 Date: Nov. 22, 2024

Related U.S. Application Data
(60) Provisional application No. 63/345,678, filed on May 25, 2022.
(60) Provisional application No. 63/447,807, filed on Feb. 23, 2023.

(51) Int. Cl. C12N 5/077; A61K 31/416; A61K 31/444

(52) U.S. Cl. CPC C12N 5/0659; A61K 31/416; A61K 31/444; C12N 2501/727

(57) ABSTRACT

The disclosure relates to compositions and methods for enhancing asymmetric division of satellite cells and promoting muscle cell/tissue regeneration, for treatment of muscle tissue injuries and muscle diseases, including muscular dystrophies.

### CROSS REFERENCE TO RELATED APPLICATIONS

This application claims priority to U.S. Provisional Patent Application No. 63/345,678 filed May 25, 2022 and titled “Modulation of Satellite Cell Polarity and Asymmetric Cell Division” and to U.S. Provisional Patent Application No. 63/447,807 filed Feb. 23, 2023 and titled “Modulation of Satellite Cell Polarity and Asymmetric Cell Division,” which are incorporated herein by reference in their entirety.

### STATEMENT REGARDING SEQUENCE LISTING

The Sequence Listing XML associated with this application is provided in XML file format and is hereby incorporated by reference into the specification. The name of the XML file containing the Sequence Listing XML is STLS_016_02WO_ST26.xml. The XML file is 28,436 bytes, and created on May 24, 2023, and is being submitted electronically via USPTO Patent Center.

### FIELD OF INVENTION

The disclosure relates to compositions and methods for enhancing asymmetric division of satellite cells and promoting muscle cell/tissue regeneration, including dosage amounts and dosage regimens, for treatment of muscle tissue injuries and muscle diseases, including muscular dystrophies.

### BACKGROUND

Stem cells are undifferentiated or immature cells capable of giving rise to multiple specialized cell types and ultimately, to terminally differentiated cells. Most adult stem cells are lineage-restricted and are generally referred to by their tissue origin. Unlike any other cells, stem cells are able to renew themselves to generate a virtually endless supply of mature cell types when needed over the lifetime of an organism).

Satellite cells are a heterogeneous population of stem cells and small mononuclear progenitor cells found in mature muscle tissue (Kuang et al. Cell, 2007. 129 (5): p. 999-1010).

Satellite cells express a number of genetic markers, including the paired-box transcription factor Pax7, which plays a central regulatory role in satellite cell function and survival and can be used as a marker of satellite cells (Kuang et al., 2006. J. Cell Biol. 172 (1): 103-13; Seale et al., 2000. Cell. 102 (6): 777-86). The satellite cell population is composed of subpopulations of stem cells (Pax7+/Myf−) and committed myogenic progenitors (Pax7+/Myf5+). Pax7+/Myf5− satellite cells give rise to Pax7+/Myf5+ satellite cells through basal-apical oriented asymmetric cell division within the satellite cell niche. Pax7+/Myf5+ satellite cells preferentially differentiate, whereas Pax7+/Myf5-satellite cells extensively contribute to the satellite cell compartment. This asymmetric satellite cell division helps skeletal muscle maintain its stem cell pool and at the same time contribute to muscle regeneration.

Satellite cells are involved in the normal growth of muscle, as well as the regeneration of injured or diseased muscle tissue. In undamaged muscle, the majority of satellite cells are quiescent, and do not differentiate or undergo cell division. However, upon muscle damage, such as physical trauma or strain, repeated exercise, or in disease, satellite cells become activated, proliferate, and give rise to a population of transient amplifying progenitors, which are myogenic precursors cells (myoblasts) expressing myogenic regulatory factors (MRF), such as MyoD and Myf5. During muscle regeneration, myoblasts undergo multiple rounds of division before committing to terminal differentiation, fusing with the host fibers or generating new myofibers to reconstruct damaged tissue (Charge and Rudnicki, 2004. Physiol. Rev. 84 (1): 209-38).

Although skeletal muscle has regenerative capacity, this ability is significantly impaired in certain muscle diseases, such as Duchenne muscular dystrophy (DMD). Specifically, skeletal muscle stem cells (also known as satellite cells) have cell-intrinsic defects in DMD that result in significantly reduced numbers of asymmetric division. There is clearly a need in the art for muscle disease treatments that address the problem of reduced asymmetric divisions of satellite cells, as well as methods for the generation of cells that are capable of producing functional muscle.

### SUMMARY OF THE INVENTION

The present disclosure provides compositions and methods, including dosage amounts and dosage regimens, that may be used to increase or promote asymmetric cell division and treat a variety of diseases and disorders, including muscular dystrophies.

In one aspect, the disclosure provides a method for increasing asymmetric cell division of skeletal muscle stem cells, the method comprising contacting the skeletal muscle stem cells with an inhibitor of any of adaptor-associated kinase 1 (AAK1), cyclin G-associated kinase (GAK), or myristoylated and/or palmitoylated serine/threonine kinase 1 (MPSK1, also known as STK16). In certain embodiments, the skeletal muscle stem cells are damaged or injured skeletal muscle stem cells or are present within damaged or injured skeletal muscle tissue. In some embodiments, the muscle tissue is damaged or injured as a result of: physical injury or accident, disease, gene mutation, infection, over-use, loss of blood circulation, muscle atrophy, muscle wasting, dystrophic muscle, or ageing. In some embodiments, the skeletal muscle stem cells are diseased skeletal muscle stem cells comprising a mutation associated with a muscular dystrophy, optionally Duchenne muscular dystrophy (DMD), Becker muscular dystrophy (BMD), Emery-Dreifuss muscular dystrophy, Landouzy-Dejerine muscular dystrophy, facioscapulohumeral muscular dystrophy (FSH), Limb-Girdle muscular dystrophies, von Graefe-Fuchs muscular dystrophy, oculopharyngeal muscular dystrophy (OPMD), Myotonic dystrophy (Steinert's disease) or a congenital muscular dystrophy.

In some embodiments, wherein the damaged or injured muscle stem cells comprise a mutation of a dystrophin gene. In some embodiments, the skeletal muscle stem cells are present within injured muscle tissue. In some embodiments, the skeletal muscle stem cells have reduced asymmetric cell division as compared to normal, healthy skeletal muscle stem cells.

In another aspect, the disclosure provides methods for increasing skeletal muscle tissue growth or regeneration in a subject, comprising administering to the subject an inhibitor of AAK1, GAK, or MPSK1. In some embodiments, the subject has damaged or injured skeletal muscle tissue. In some embodiments, the skeletal muscle tissue is damaged or injured as a result of: physical injury or accident, disease, gene mutation, infection, over-use, loss of blood circulation, muscle atrophy, muscle wasting, dystrophic muscle, or ageing. In some embodiments, the subject has a muscular dystrophy, optionally Duchenne muscular dystrophy (DMD), Becker muscular dystrophy (BMD), Emery-Dreifuss muscular dystrophy, Landouzy-Dejerine muscular dystrophy, facioscapulohumeral muscular dystrophy (FSH), Limb-Girdle muscular dystrophies, von Graefe-Fuchs muscular dystrophy, oculopharyngeal muscular dystrophy (OPMD), Myotonic dystrophy (Steinert's disease) or a congenital muscular dystrophy. In some embodiments, the subject comprises a mutation of a dystrophin gene. In some embodiments, skeletal muscle stem cells within the skeletal muscle tissue have reduced asymmetric cell division as compared to normal, healthy skeletal muscle stem cells. In some embodiments, the inhibitor of AAK1, GAK, or MPSK1 does not substantially inhibit proliferation or cell cycle progression of the subject's skeletal muscle stem cells. In some embodiments, the method increases skeletal muscle tissue regeneration in the subject. In some embodiments, the subject is a mammal, optionally a human. In some embodiments, the inhibitor of AAK1, GAK, or MPSK1 is administered to the subject systemically or locally, optionally at a site of tissue damage or injury.

In another aspect, the disclosure provides methods for treating a muscular dystrophy, comprising administering to a subject in need thereof an inhibitor of AAK1, GAK, or MPSK1. In some embodiments, the subject has a muscular dystrophy selected from the group consisting of: Duchenne muscular dystrophy (DMD), Becker muscular dystrophy (BMD), Emery-Dreifuss muscular dystrophy, Landouzy-Dejerine muscular dystrophy, facioscapulohumeral muscular dystrophy (FSH), Limb-Girdle muscular dystrophies, von Graefe-Fuchs muscular dystrophy, oculopharyngeal muscular dystrophy (OPMD), Myotonic dystrophy (Steinert's disease) and a congenital muscular dystrophy. In some embodiments, the subject comprises a mutation of a dystrophin gene. In some embodiments, skeletal muscle stem cells within the subject have reduced asymmetric cell division as compared to normal, healthy skeletal muscle stem cells. In some embodiments, the inhibitor of AAK1, GAK, or MPSK1 does not substantially inhibit proliferation or cell cycle progression of the subject's skeletal muscle stem cells. In some embodiments, the method increases skeletal muscle tissue regeneration in the subject. In some embodiments, the subject is a mammal, optionally a human. In some embodiments, the inhibitor of AAK1, GAK, or MPSK1 is administered to the subject systemically or locally, optionally at a site of tissue damage or injury.

In particular embodiments of any of the methods disclosed herein, the inhibitor inhibits expression of AAK1, GAK, or MPSK1, optionally by inhibiting transcription, translation, post-translational modification, or stability of the protein component, or the gene encoding the protein component. In some embodiments, the inhibitor binds to a polynucleotide sequence that regulates expression of AAK1, GAK, or MPSKI, optionally wherein the nucleotide sequence is present within the AAK1, GAK, or MPSKI gene. In some embodiments, the inhibitor binds to a polynucleotide sequence that encodes AAK1, GAK, or MPSK1, or a polynucleotide sequence complementary to the polynucleotide sequence that encodes AAK1, GAK, or MPSK1, optionally wherein the polynucleotide sequence is present within the AAK1 gene or mRNA. In some embodiments, the polynucleotide sequence is DNA or RNA. In some embodiments, the inhibitor comprises a polynucleotide sequence. In some embodiments, the inhibitor comprises a DNA polynucleotide sequence and/or an RNA polynucleotide sequence. In some embodiments, the inhibitor comprises a shRNA, a microRNA, a gRNA, an siRNA, an aptamer, or an antisense oligonucleotide. In some embodiments, the inhibitor comprises a guide RNA targeting the AAK1 gene and a polynucleotide sequence encoding a CRISPR-Cas protein.

In particular embodiments of any of the methods disclosed herein, the inhibitor inhibits an activity of AAK1, GAK, or MPSK1. In some embodiments, the inhibitor binds to AAK1, GAK, or MPSK1. In some embodiments, the inhibitor comprises a polypeptide. In some embodiments, the inhibitor comprises an antibody, or a functional fragment thereof, that binds to AAK1, GAK, or MPSK1. In some embodiments, the inhibitor is an organic molecule, e.g., a small organic molecule. In some embodiments, the inhibitor is selected from the group consisting of: SGC-AAK1-1, LP-935509, LP-922761, BMT-090605, BMT-124110, LP-927443, and BMS-901715. In some embodiments, the inhibitor inhibits AAK1, GAK, or MPSK1 kinase activity or AAK1, GAK, or MPSK1 ATP binding activity. In some embodiments, the inhibitor of AAK1, GAK, or MPSK1 does not substantially inhibit proliferation or cell cycle progression of the skeletal muscle stem cells. In some embodiments, the inhibitor inhibits AAK1. In some embodiments, the inhibitor inhibits GAK. In some embodiments, the inhibitor inhibits MPSK1.

In particular embodiments of any of the methods disclosed herein, the contacting between the inhibitor and the cells occurs in vitro, in vivo, ex vivo, or in situ. In some embodiments, the cells are mammalian, optionally human.

In particular embodiments of any of the methods disclosed herein, the inhibitor is administered once every 1, 2, 3, 4, 5, 6, or 7 days. In some embodiments, the inhibitor is administered once about every 3 days. In some embodiments, the inhibitor is administered 1, 2, 3, 4, 5, 6, or 7 times per week. In some embodiments, the inhibitor is administered 2 times per week.

In particular embodiments of any of the methods disclosed herein, the inhibitor is administered at a dose of about 0.01 mg/kg to about 300 mg/kg. In some embodiments, the inhibitor is administered at a dose of about 0.1 mg/kg to about 20 mg/kg. In some embodiments, the inhibitor is administered at a dose of about 0.1, about 0.3, about 0.7, about 1, about 2, about 3, about 4, about 5, about 6, about 7, about 8, about 9, about 10, about 11, about 12, about 13, about 14, about 15, about 16, about 17, about 18, about 19 or about 20 mg/kg. In some embodiments, the inhibitor is administered at a dose of about 1 mg/kg.

On other aspects, any of the methods disclosed herein are performed using an inhibitor of STK38 or STK38L.

### BRIEF DESCRIPTION OF THE DRAWINGS

FIGS. 1A and 1B are graphs showing the effects of AAK1 inhibition on asymmetric muscle stem cell division. FIG. 1A shows the proportion of asymmetric stem cell divisions (%) in cells treated with an siRNA targeting AAK1 or a control siRNA targeting CTRL. FIG. 1B shows the % of MyoG positive cells following treatment with the siRNA targeting AAK1 or the control siRNA targeting CTRL.

FIGS. 2A and 2B are graphs showing the effects of AAK1 inhibition on muscle stem cell cycle progression. FIG. 2A shows the total number of satellite cells per fiber at 42 h following treatment with an siRNA targeting AAK1 or a control siRNA targeting CTRL. FIG. 2B shows the number of myogenic cells per fiber following treatment with the siRNA targeting AAK1 or the control siRNA targeting CTRL.

FIGS. 3A-3D are graphs showing the effects of AAK1 inhibition on asymmetric muscle stem cell division. FIG. 3A shows the total number of satellite cells per fiber at 42 hours following treatment with 0.1% DMSO (1; negative control), 100 nM SGC-AAK1-1 (2), or 100 nM LP-935509 (3). FIG. 3B shows the percentage of YFP-negative satellite stem cells following treatment with 0.1% DMSO (1; negative control), 100 nM SGC-AAK1-1 (2), or 100 nM LP-935509 (3). FIG. 3C shows the number of symmetric satellite stem cell divisions per fiber following treatment with 0.1% DMSO (1; negative control), 100 nM SGC-AAK1-1 (2), or 100 nM LP-935509 (3). FIG. 3D shows the number of asymmetric satellite stem cell divisions per fiber following treatment with 0.1% DMSO (1; negative control), 100 nM SGC-AAK1-1 (2), or 100 nM LP-935509 (3). NS=not significant.

FIGS. 4A-F show structures of illustrative AAK1 inhibitors.

FIGS. 5A-D are graphs showing the effects of AAK1 inhibition on asymmetric muscle stem cell division. FIG. 5A shows the total number of satellite cells per fiber at 42 hours following treatment with 0.1% DMSO (1; negative control), 100 nM SGC-AAK1-1 (2), or 100 nM LP-935509 (3). FIG. 5B shows the percentage of YFP-negative satellite stem cells following treatment with 0.1% DMSO (1; negative control), 100 nM SGC-AAK1-1 (2), or 100 nM LP-935509 (3). FIG. 5C shows the number of symmetric satellite stem cell divisions per fiber following treatment with 0.1% DMSO (1; negative control), 100 nM SGC-AAK1-1 (2), or 100 nM LP-935509 (3). FIG. 5D shows the proportion of asymmetric satellite stem cell divisions per fiber following treatment with 0.1% DMSO (1; negative control), 100 nM SGC-AAK1-1 (2), or 100 nM LP-935509 (3). NS=not significant.

FIGS. 6A-D provide graphs showing the effect on EDL fibers, in terms of total number of satellite cells per fiber (FIG. 6A), number of asymmetric satellite stem cell divisions (FIG. 6B), proportion of asymmetric satellite stem cell divisions (FIG. 6C), and number of YFP+ satellite committed cells per fiber (FIG. 6D) after 42 hour treatment with siRNA targeting Aak1 or Gak, or a control siRNA.

FIGS. 7A-E provides graphs showing the effect on EDL fibers, in terms of total number of satellite cells per fiber (FIG. 7A), % YFP-satellite stem cells (FIG. 7B), rate of YFP-divisions, calculated as the #of YPF-cells in doublets/ #of YFP-single cells (FIG. 7C), rate of YFP+ divisions, calculated as the #of YFP+ cells in doublets/ #of YFP+ single cells (FIG. 7D), and #of YFP+ satellite committed cells per fiber (FIG. 7E) after 42 hour treatment with siRNA targeting Aak1 or Gak, or a control siRNA.

FIGS. 8A-D provides graphs showing the effect on EDL fibers, in terms of #of symmetric satellite stem cell divisions per fiber (FIG. 8A), #of asymmetric satellite stem cell divisions per fiber (FIG. 8B), proportion of asymmetric stem cell divisions (FIG. 8C), and #of symmetric satellite committed cells per fiber (FIG. 8D), after 42 hour treatment with siRNA targeting Bmp2k, MPSK1, Numb, or a control siRNA.

FIGS. 9A-D provides graphs showing the effect on EDL fibers, in terms of total number of satellite cells per fiber (FIG. 9A), % YFP-satellite stem cells (FIG. 9B), rate of YFP-divisions, calculated as the #of YPF-cells in doublets/ #of YFP-single cells (FIG. 9C), and rate of YFP+ divisions, calculated as the #of YFP+ cells in doublets/ #of YFP+ single cells (FIG. 9D) after 42 hour treatment with siRNA targeting Bmp2k, Mpsk1, Numb, or a control siRNA.

FIG. 10 shows a schematic overview of an in vivo experiment assessing the effect of AAK1 inhibition on asymmetric muscle cell division, in which C57BL/10ScSn-Dmd^{mdx}/J (mdx) mice received cardiotoxin (CTX)-induced injury followed by treatment with DMSO (vehicle control) or LX9211 (IP #1, IP #2; SAT3003).

FIGS. 11A-D shows the effects of in vivo AAK1 inhibition (as in FIG. 10) on asymmetric muscle stem cell division. Graphs are provided showing quantification of % of Pax7+ cells out of total DAPI+ cells (FIG. 11A), % of MyoG+ cells out of total DAPI+ cells (FIG. 11B), and % of MyoG+ cells out of total myogenic cells (FIG. 11C), as measured by single cell assay.

FIG. 11D shows distribution of minimum Feret of myofibers in regenerating TA muscles of mdx mice 10 days post cardiotoxin-induced injury and treated with DMSO (vehicle control), or LX9211 (SAT3003), as determined by histology. At the 40 um point on the x-axis, the lines are, in order from top to bottom, SAT3003 then Control. The error bars represent means=SEM; p-values: *=<0.05; **=<0.01.

FIG. 12 shows a schematic overview of treatment with LX9211 (SAT3003) according to specified dosage regimens in C57BL/10ScSn-Dmd^{mdx}/J (mdx) mice. Groups of 5 mice each received one of the following dosage regimens: 2×: SAT3003 at 1 mg/kg on day 0 and 3 (2× per week for first week only); 4×: SAT3003 at 1 mg/kg on day 0, 3, 7 and 10; 7×: SAT3003 at 1 mg/kg on day 0, 3, 5, 7, 10, 12, 14, and 17; or DMSO only (vehicle control) on day 0, 3, 5, 7, 10, 12, 14, and 17.

FIGS. 13A-D shows the effects of in vivo AAK1 inhibition according to specified dosage regimens on asymmetric muscle stem cell division. Graphs are provided showing quantification of TA area (FIG. 13A), percentage of cells that are MyoG+ (FIG. 13B), and the density of MyoG+ (FIG. 13C) and Pax7+ (FIG. 13D) cells in TA muscles of mdx mice treated with DMSO (vehicle control) or LX9211 (SAT3003) as in FIG. 12, as measured by histology. The error bars represent means SEM; p-values: *=<0.05; **=<0.01.

FIGS. 14A-E shows the effects of in vivo AAK1 inhibition according to specified dosage regimens on asymmetric muscle stem cell division. Graphs are provided showing the total fiber area (FIG. 14A), average Feret diameter (FIG. 14B) and the distribution of minimum Feret of myofibers in TA muscles of mdx mice treated with DMSO (vehicle control) or LX9211 (SAT3003) according to the 2× (FIG. 14C), 4× (FIG. 14D), or 7× (FIG. 14E) dosage regimens as in FIG. 12. The lines are, in order from top to bottom: SAT3003 then Control to the right of the 40 μm point of the x-axis (FIG. 14C and FIG. 14D); SAT3003 (7×) then Control at the 60 μm point of the x-axis (FIG. 14E). The error bars represent means±SEM; p-values: *=<0.05; **=<0.01.

FIG. 15 shows a schematic overview of treatment with specific dosage amounts of LX9211 (SAT3003) in C57BL/10ScSn-Dmd^{mdx}/J (mdx) mice. Groups of 7 mice each received one of the following dosage amounts: (1) SAT3003 at 0.1 mg/kg, (2) SAT3003 at 0.3 mg/kg, (3) SAT3003 at 1 mg/kg, or DMSO only (vehicle control). Doses were administered on days 0, 3, 7, and 10.

FIGS. 16A-D show the effects of in vivo AAK1 inhibition with specific dosage amounts of LX9211 (SAT-3003) on TA muscle strength, as measured by maximum tetanic force (nM). Graphs are provided showing the maximum tetanic force of TA muscles of mdx mice treated with 0.1 mg/kg (FIG. 16A), 0.3 mg/kg (FIG. 14B), or 1 mg/kg (FIG. 14C) of LX9211 (SAT-3003) as in FIG. 15, as a percentage increase over vehicle control. FIG. 16D shows all four groups in the same graph. The error bars represent means±SEM; p-values (one-tailed t-tests) are shown in FIG. 16A-C.

FIG. 17A-D show the effects of in vivo AAK1 inhibition with specific dosage amounts of LX9211 (SAT-3003) on TA muscle strength, as measured by specific force (mN/mm2). Graphs are provided showing the specific force of TA muscles of mdx mice treated with 0.1 mg/kg (FIG. 17A), 0.3 mg/kg (FIG. 17B), or 1 mg/kg (FIG. 17C) of LX9211 (SAT-3003) as in FIG. 15, as a percentage increase over vehicle control. FIG. 17D shows all four groups in the same graph. The error bars represent means±SEM; p-values (one-tailed t-tests) are shown in FIGS. 17A-C.

FIGS. 18A-D show the effects of in vivo AAK1 inhibition with specific dosage amounts of LX9211 (SAT-3003) on TA muscle strength, as measured by twitch force (mN). Graphs are provided showing the twitch force of TA muscles of mdx mice treated with 0.1 mg/kg (FIG. 18A), 0.3 mg/kg (FIG. 17B), or 1 mg/kg (FIG. 17C) of LX9211 (SAT-3003) as in FIG. 15, as a percentage increase over vehicle control. FIG. 18D shows all four groups in the same graph. The error bars represent means±SEM; p-values (one-tailed t-tests) are shown in FIGS. 18A-C.

### DETAILED DESCRIPTION

The disclosure provides compositions and methods for modulating cell signalling pathways and increasing asymmetric division of satellite cells, e.g., to increase or enhance muscle regeneration, or as a therapeutic strategy for a variety of muscle wasting diseases, such as, but not limited to, Duchenne muscular dystrophy (DMD). The methods disclosed herein may similarly be used to increase or stimulate myofiber and/or muscle tissue regeneration.

Adult skeletal muscle has regenerative capability. For example, after acute muscle injury, new muscle fibers form within about a week as the result of expansion and differentiation of self-renewing muscle satellite cells. During regeneration, normally quiescent satellite cells are activated to produce daughter myogenic precursor cells, which then form the new muscle fibers. However, the number of satellite cells decreases during ageing, which results in reduced muscle regeneration ability. Additionally, in certain muscle diseases, such as DMD, decreased satellite cell regenerative ability and number results in impaired regeneration and accelerated disease progression.

Satellite cells are a heterogeneous population primarily composed of committed progenitors, together with a small population of muscle stem cells that are capable of long-term self-renewal. Satellite cells undergo two forms of cell division: asymmetric division, in which a major subpopulation of cells generates daughter cells committed to myogenic differentiation, while a small subpopulation of cells give rise to self-renewing daughter cells; and symmetric division, in which one stem cell population generates two identical stem daughter cells. In regenerating muscle, satellite cell symmetric divisions occur mostly in a planar orientation (parallel to the myofiber), whereas asymmetric divisions occur in an apicobasal orientation (perpendicular to the myofiber). Thus, in the context of acute muscle injury, asymmetric satellite cell division generates a stem cell and a transient-amplifying progenitor capable of dividing multiple times to generate a cohort of myogenic precursor cells that differentiate by fusion with existing myofibers or by forming new myofibers, whereas symmetric satellite cell division promotes expansion of satellite stem cells and maintains homeostasis of the stem cell compartment.

Satellite cells are juxtaposed against the myofiber sarcolemma within a cleft that forms the niche beneath the basal lamina. Quiescent satellite cells are polarized and express different adhesion proteins on the basal versus the apical cell surface, which influence quiescence and cell polarity. In healthy satellite cells, dystrophin act as a scaffolding protein during mitosis to bind Parlb, leading to asymmetric segregation of Pard3 and the PAR complex and apical-basal orientation of the centrosomes prior to mitotic division. Following apical-basal oriented asymmetric division, the committed daughter cell no longer has contact with the basal lamina, while the stem cell in contact with the basal lamina maintains niche interactions to promote a return to quiescence.

### Abbreviations

As used in this specification and the appended claims, the singular forms “a,” “an” and “the” include plural references unless the content clearly dictates otherwise.

As used in this specification, the term “and/or” is used in this disclosure to either “and” or “or” unless indicated otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Decrease” or “inhibit” may refer to a decrease or inhibition of at least 5%, for example, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or 100%, for example, as compared to a reference or control level, e.g., in control cells or tissue.

“Increase” may refer to an increase of at least 5%, for example, at least 5%, at least 6%, at least 7%, at least 8%, at least 9%, at least 10%, at least 15%, at least 20%, at least 25%, at least 30%, at least 35%, at least 40%, at least 45v, at least 50%, at least 55%, at least 60%, at least 65%, at least 70%, at least 75%, at least 80%, at least 85%, at least 90%, at least 95%, at least 99% or at least 100%, for example, as compared to a reference level or the level in control cells or tissue. Increase also means increases of at least 1-fold, for example, at least 1-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 30-fold, at least 40-fold, at least 50-fold, at least 60-fold, at least 70-fold, at least 80-fold, at least 90-fold, at least 100-fold, at least 200-fold, at least 500-fold, at least 1000-fold or more, for example, as compared to the level of a reference or the level in control cells or tissue.

The term “inhibitor” may refer to any agent that inhibits the expression or activity of a target gene, mRNA and/or protein in a cell, tissue, organ, or subject. The expression level or activity of target mRNA and/or protein in a cell may be reduced via a variety of means, including but not limited to reducing the total amount of target protein or inhibiting one or more activity of the target protein. In various embodiments, an inhibitor may inhibit the expression of a target gene, target mRNA, or a target protein, and/or an inhibitor may inhibit a biological activity of a target protein. In certain embodiments, the biological activity is kinase activity. For example, an inhibitor may competitively bind to the ATP-binding site of a kinase and inhibit its kinase activity, or it may allosterically block the kinase activity. In certain embodiments, an inhibitor causes increased degradation of a target protein. Methods for determining the expression level or the activity of a target gene or polypeptide are known in the art and include, e.g., RT-PCR and FACS. Methods for determining kinase activity are known in the art, including e.g., those described in Nat Methods (2005), 2 (1): 17-25.doi: 10.1038/nmeth731.

“Subjects” includes animals (e.g., mammals, swine, fish, birds, insects etc.). In some embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals such as dogs and cats. In some embodiments (e.g., particularly in research contexts) subjects are rodents (e.g., mice, rats, hamsters), rabbits, primates, or swine such as inbred pigs and the like. The terms “subject” and “patient” are used interchangeably herein.

“Tissue” is an ensemble of similar cells from the same origin that together carry out a specific function, e.g., smooth muscle tissue or skeletal muscle tissue.

An “antibody” is an immunoglobulin (Ig) molecule capable of specific binding to a target, such as a carbohydrate, polynucleotide, lipid, or polypeptide, through at least one epitope recognition site, located in the variable region of the Ig molecule. As used herein, the term encompasses not only intact polyclonal or monoclonal antibodies, but also fragments thereof, such as dAb, Fab, Fab′, F(ab′) 2, Fv, single chain (scFv), synthetic variants thereof, naturally occurring variants, fusion proteins comprising an antibody portion with an antigen-binding fragment of the required specificity, chimeric antibodies, nanobodies, and any other modified configuration of the immunoglobulin molecule that comprises an antigen-binding site or fragment of the required specificity.

“Fragment” refers to a portion of a polypeptide or polynucleotide molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. A “functional fragment” of an antibody is a fragment that maintains one or more activities of the antibody, e.g., it binds the same epitope and or possesses a biological activity of the antibody. In particular embodiments, a functional fragment comprises the six CDRs present in the antibody.

“Pharmaceutical compositions” include compositions of one or more inhibitors disclosed herein and one or more pharmaceutically acceptable carrier, excipient, or diluent.

“Pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable carrier” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, and/or emulsifier which has been approved by the United States Food and Drug Administration as being acceptable for use in humans and/or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations. Except insofar as any conventional media and/or agent is incompatible with the agents of the present disclosure, its use in therapeutic compositions is contemplated. Supplementary active ingredients also can be incorporated into the compositions.

“Dose” means a specified quantity of a pharmaceutical agent provided in a single administration, or in a specified time period. In certain embodiments, a dose can be administered in two or more boluses, tablets, or injections. In certain embodiments, a dose can be administered in two or more injections to minimize injection site reaction in an individual. Doses can be stated as the amount of pharmaceutical agent per hour, day, week or month. “Dosage amount” may be used interchangeably with “dose”.

“Dosage regimen” means a schedule according to which doses of a pharmaceutical agent are provided, e.g., daily, weekly, or other schedule capable of being developed by a person of ordinary skill in the art.

“Effective amount” as used herein refers to an amount of an agent effective in achieving a particular effect, e.g., increasing asymmetric cell division or tissue regeneration in a cell, tissue, organ or subject. In certain embodiments, the increase is at least 10%, at least 20%, at least 30%, at least 40%, at least 50%, at least 60%, or at least 70%, as compared to the amount prior to or without treatment. In the context of therapeutic treatment of a subject, an effective amount may be, e.g., an amount effective or sufficient to reduce one or more disease symptoms in the subject, e.g., a subject with a muscular dystrophy.

“Effective concentration” as used herein refers to the minimum concentration (mass/volume) of an agent and/or composition required to result in a particular physiological effect. As used herein, effective concentration typically refers to the concentration of an agent required to increase, activate, and/or enhance a particular physiological effect.

### Methods

As described in the accompanying Examples, the disclosure identifies targets useful in increasing asymmetric division and promoting muscle tissue regeneration. Accordingly, the disclosure provides methods and compositions for increasing asymmetric cell division, e.g., of satellite cells, promoting or increasing muscle cell and tissue proliferation and regeneration, and treating diseases, disorders and injuries that would benefit from muscle tissue generation.

Asymmetric cell division is a type of cell division that produces two different, non-identical daughter cells, typically with different properties or cellular fates, through the unequal inheritance or distribution of cell fate determinants, e.g., cellular proteins and RNAs. Asymmetric satellite cell division (and modulation thereof) may be determined or measured according to known methods in the art, and according to methods disclosed herein, e.g., using cultured muscle fibers.

Muscle fiber and tissue regeneration refers to generation of new muscle fiber and tissue, typically as the result of expansion and differentiation of self-renewing muscle satellite cells. During the regeneration process, normally quiescent satellite cells are activated to produce daughter myogenic precursor cells, which then form new muscle fibers, which may fuse with existing muscle fibers to generate new muscle tissue. Generation of muscle fiber and tissue may be determined or measured according to known methods in the art, and according to methods disclosed herein, e.g., an animal model to determine prevalence and/or density or mass of MyoG+ progenitor cells, muscle fiber area, myofiber Feret diameter, and/or muscle strength.

In one embodiment, the disclosure provides a method for increasing asymmetric cell division of stem cells or any other cell that undergoes asymmetric cell division, e.g., skeletal muscle stem cells, or satellite cells, the method comprising contacting the cells with an inhibitor of AAK1, GAK, or MPSK1. In particular embodiments, the inhibitor inhibits AAK1. In particular embodiments, the stem cells are muscle stem cells, retinal stem cells, neural stem cells, hematopoietic stem cells, intestinal stem cells, epidermal stem cells, or cancer or tumor stem cells. In certain embodiments, the stem cells are muscle stem cells or satellite cells.

In another embodiments, the disclosure provides a method for increasing skeletal muscle tissue growth or regeneration, comprising contacting skeletal muscle stem cells with an inhibitor of AAK1, GAK, or MPSK1. In particular embodiments, the inhibitor inhibits AAK1.

Methods disclosed herein may be practiced in vitro, ex vivo, or in vivo. For example, the methods may be used to promote growth and proliferation of satellite cells in vitro, to generate tissue, e.g., muscle tissue, or to treat a subject in need of increased muscle tissue generation.

In certain embodiments, methods are practiced in vitro or ex vivo, e.g., to promote or increase asymmetric cell division of stem cells. Such methods may be used, e.g., to generate tissue models or organoids. In addition, such methods may be used, e.g., to produce progenitor cells. In vitro and ex vivo tissues, organoids, and progenitor cells have a variety of uses, including, e.g., use in research and use in screening potential therapeutic drug candidates.

In certain embodiments of any of the methods disclosed herein, the stem cells, e.g., skeletal muscle stem cells, are damaged or injured stem cells or are present within damaged or injured tissue, e.g., skeletal muscle tissue. In certain embodiments, the stem cells or tissue is damaged or injured as a result of physical injury or accident, disease, gene mutation, infection, over-use, loss of blood circulation, muscle atrophy, cachexia, muscle wasting, dystrophic muscle, or cytopenia or ageing. However, the stem cells, e.g., skeletal muscle stem cells, may also be healthy or present withing healthy tissue. In certain embodiments, the stem cells have reduced asymmetric cell division as compared to normal, healthy stem cells. However, the stem cells, e.g., skeletal muscle stem cells, may have comparable asymmetric cell division as compared to normal healthy stem cells.

In many diseases and conditions affecting muscle, there is a reduction in muscle mass associated with reduced numbers of satellite cells and a reduced ability of the satellite cells to repair, regenerate, and grow skeletal muscle. Illustrative diseases and conditions affecting muscle include wasting diseases, such as cachexia, muscular attenuation or atrophy, including sarcopenia, ICU-induced weakness, surgery-induced weakness (e.g., following knee or hip replacement), and muscle degenerative diseases, such as muscular dystrophies, and any of these disease and conditions can be treated according to the disclosed methods. In certain embodiments, the stem cells or tissue are muscle stem cells, e.g., satellite cells, or muscle tissue damaged or injured due to muscle wasting or atrophy, for example, cancer-related cachexia.

In certain embodiments, the stem cells are diseased skeletal muscle stem cells comprising a mutation associated with a muscular dystrophy, optionally Duchenne muscular dystrophy or Becker muscular dystrophy. In particular embodiments, the damaged or injured muscle stem cells comprise a mutation of a dystrophin gene.

In particular embodiments, the disclosure provides methods to treat a muscular dystrophy, comprising administering to a subject diagnosed with or suspected of having a muscular dystrophy an inhibitor disclosed herein, e.g., an AAK1 inhibitor. Muscular dystrophies are genetic diseases characterized by progressive weakness and degeneration of the skeletal or voluntary muscles which control movement. The muscles of the heart and some other involuntary muscles are also affected in some forms of muscular dystrophy. In many cases, the histological picture shows variation in fiber size, muscle cell necrosis and regeneration, and often proliferation of connective and adipose tissue. The progressive muscular dystrophies include at least Duchenne muscular dystrophy (DMD), Becker muscular dystrophy (BMD), Emery-Dreifuss muscular dystrophy, Landouzy-Dejerine muscular dystrophy, facioscapulohumeral muscular dystrophy (FSH), Limb-Girdle muscular dystrophies, von Graefe-Fuchs muscular dystrophy, oculopharyngeal muscular dystrophy (OPMD), Myotonic dystrophy (Steinert's disease) and congenital muscular dystrophies. Any of these diseases and/or related symptoms may be treated or improved according to the methods disclosed herein.

Generally, cells/tissues/subjects are contacted with or administered an effective amount of the inhibitor of AAK1, GAK, or MPSK1, e.g., an AAK1 inhibitor. The effective amount may be an amount or concentration effective to increase asymmetric cell division, promote cell growth or tissue regeneration, or treat a disease or disorder, including any of those disclosed herein.

In some embodiments, an inhibitor may be administered at a dosage from about 0.01 mg/kg to about 300 mg/kg. As used herein, mg/kg refers to the amount of inhibitor administered (in mg) relative to the weight of the subject (in kg) to which it is administered. In another embodiment, an inhibitor may be administered at a dosage from about 0.1 mg/kg to about 20 mg/kg. For example, the inhibitor may be administered to a subject at a dosage of about 0.01, 0.03, 0.07, 0.1, 0.3, 0.7, 1, 2, 3, 4, 5, 6, 7, 8, 9, 10, 11, 12, 13, 14, 15, 16, 17, 18, 19 or 20 mg/kg, or within a range between any of the proceeding values, for example, between about 10 mg/kg and about 15 mg/kg, between about 6 mg/kg and about 12 mg/kg, between 0.1 and 2 mg/kg, and the like. In another embodiment, an inhibitor is administered at a dosage of ≤15 mg/kg. In a preferred embodiment, the inhibitor is administered at a dosage of about 1 mg/kg. For example, an inhibitor may be administered at 1 mg/kg per day for 7 days for a total of 7 mg/kg per week. For example, a compound may be administered at 10 mg/kg twice per day for 7 days for a total of 140 mg/kg per week.

In many embodiments, the dosages described herein may refer to a single dosage, a daily dosage, or a weekly dosage. In one embodiment, an inhibitor may be administered once per day. In another embodiment, a compound may be administered twice per day. In some embodiments, an inhibitor may be administered three times per day. In some embodiments, an inhibitor may be administered four times per day. In some embodiments, an inhibitor may be administered 1, 2, 3, 4, 5, 6, 7, 8, 9, 10, 11, 12, 13, 14, 15, 16, 17, 18, 19, 20, 21, 22, 23, or 24 times per week. In a preferred embodiment, the inhibitor may be administered two times a week. In other embodiments, the inhibitor is administered once biweekly. In additional embodiments, the inhibitor is administered once every 1, 2, 3, 4, 5, 6, or 7 days. In a preferred embodiment, the inhibitor is administered once about every 3 days. In a preferred embodiment, the inhibitor is administered once about every 4 days.

In some embodiments, the period during which the inhibitor is administered (“treatment period”) may be 1, 2, 3, 4, 5, 6, 7, 8, 9, 10, 12, 13, 14, 15, 16, 17, 18, 19, 20, 21, 22, 23, or 24 weeks. In some embodiments, the treatment period may be a maximum of 4 weeks. In a preferred embodiment, the treatment period may be 2 weeks. In some embodiments, the treatment period may be a maximum of 2 weeks. In some embodiments, the treatment period may be a maximum of 3 weeks.

In some embodiments, the inhibitor may be administered a maximum of 2 or 3 times per week for a maximum of 4 weeks. In an additional preferred embodiment, the inhibitor may be administered twice a week for 2 weeks. In a preferred embodiment, the inhibitor may be administered once every about 3 days for 2 weeks.

In some embodiments, the treatment period may be followed by a period during which the inhibitor is not administered (“non-treatment period”). In some embodiments, the non-treatment period is in turn followed by a period during which treatment is resumed. In other embodiments, the treatment may not be resumed following a non-treatment period.

A wide variety of administration methods may be used in conjunction with the inhibitors according to the methods disclosed herein. For example, inhibitors may be administered or co-administered topically, orally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, intrathecally, transmucosally, pulmonary, or parenterally, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly. In some embodiments, an inhibitor may be administered orally or parenterally, e.g., intravenously or subcutaneously. In some embodiments, an inhibitor may be administered orally at a dosage of ≤15 mg/kg once per day.

The actual dosage employed may be varied depending upon the requirements of the subject and the severity of the condition being treated. The dosage regimen may be selected in accordance with a variety of factors including type, species, age, weight, sex and medical condition of the patient; the severity of the condition to be treated; the route of administration; the renal or hepatic function of the patient; and the particular compound employed. A physician or veterinarian of ordinary skill in the art can readily determine and prescribe the effective amount of the inhibitor required to prevent, counter or arrest the progress of the condition. For convenience, the total daily dosage may be divided and administered in portions during the day as required.

### Inhibitors

The accompanying Examples describe inhibition of AAK1, GAK, or MPSK1 as a target for the treatment of DMD and similar muscle wasting diseases, as well as for promoting asymmetric cell division and tissue regeneration and treating conditions that would benefit from such. As shown in the accompanying Examples, AAK1, GAK, or MPSKI may be inhibited in order to increase asymmetric cell division. Methods disclosed herein may target for inhibition AAK1, GAK, or MPSK1. In particular embodiments, the methods target AAK1 for inhibition. On the other hand, the methods may indirectly inhibit the targets identified herein by increasing expression or activity of an inhibitor of the target. In each case, the agent used may be referred to herein as an “inhibitor,” since it ultimately inhibits the target. In particular embodiments, methods disclosed herein result in decreased activity of AAK1, GAK, or MPSK1.

In certain embodiments, methods disclosed herein are performed using an inhibitor of AAK1. AAK1 is a serine/threonine kinase enzyme, and it is one of four kinases in the Numb associate kinase (NAK) family of proteins in mammals. AAK1, the adaptor-associated kinase 1, directly interacts with the membrane-tethered active form of Notch released by metalloprotease cleavage. Active AAK1 acts upstream of the γ-secretase cleavage by stabilizing both the membrane-tethered activated form of Notch and its monoubiquitinated counterpart. It is believed that AAK1 acts as an adaptor for Notch interaction with components of the clathrin-mediated pathway such as Eps15b.

In certain embodiments, methods disclosed herein are performed using an inhibitor of another member of the NAK family. The Numb-associated family of protein kinases (NAKs) constitute a diverse family in terms of Ser/Thr kinases in both their function and structure, sharing little conservation outside of the kinase domain. Humans have four known homologs: AAK1 (adaptor-associated kinase 1), BIKE/BMP2K (BMP-2-inducible kinase), GAK (cyclin G-associated kinase), and MPSK1 (myristoylated and palmitoylated serine/threonine kinase 1, also known as STK16). BIKE is structurally closely related to AAK1, plays a role in osteoblast differentiation, and has also recently been identified as a clathrin-coated vesicle-associated protein. GAK is a known association partner of cyclin G and CDK5 and among its known functions some are shared with AAK1. It is essential for clathrin trafficking and mediates binding to the plasma membrane and trans-Golgi network, as well as being required for maintenance of centrosome maturation and progression through mitosis. MPSK1 is the most distantly related of the family members and its physiological functions remain poorly understood, although it is known to be a Golgi-associated kinase with a role in the regulation of secretion in the constitutive secretory pathway at the trans-Golgi network. In addition, MPSK1 has also been linked to mammary development in mice. In particular embodiments, the methods disclosed herein are performed using an inhibitor of any one or more of AAK1, GAK, and MPSK1.

In certain embodiments, the human AAK1 protein has the following sequence of SEQ ID NO: 1 (although other isoforms are known in the art and shown below):

(SEQ ID NO: 1)
MKKFFDSRREQGGSGLGSGSSGGGGSTSGLGSGYIGRVFGIGRQQVTVDEVLAEGGFAIVFLVRTSNGM
KCALKRMFVNNEHDLQVCKREIQIMRDLSGHKNIVGYIDSSINNVSSGDVWEVLILMDFCRGGQVVNLM
NQRLQTGFTENEVLQIFCDTCEAVARLHQCKTPIIHRDLKVENILLHDRGHYVLCDFGSATNKFQNPQT
EGVNAVEDEIKKYTTLSYRAPEMVNLYSGKIITTKADIWALGCLLYKLCYFTLPFGESQVAICDGNFTI
PDNSRYSQDMHCLIRYMLEPDPDKRPDIYQVSYFSFKLLKKECPIPNVQNSPIPAKLPEPVKASEAAAK
KTQPKARLTDPIPTTETSIAPRQRPKAGQTQPNPGILPIQPALTPRKRATVQPPPQAAGSSNQPGLLAS
VPQPKPQAPPSQPLPQTQAKQPQAPPTPQQTPSTQAQGLPAQAQATPQHQQQLFLKQQQQQQQPPPAQQ
QPAGTFYQQQQAQTQQFQAVHPATQKPAIAQFPVVSQGGSQQQLMQNFYQQQQQQOOOOOOOQLATALH
QQQLMTQQAALQQKPTMAAGQQPQPQPAAAPQPAPAQEPAIQAPVRQQPKVQTTPPPAVQGQKVGSLTP
PSSPKTQRAGHRRILSDVTHSAVFGVPASKSTQLLQAAAAEASLNKSKSATTTPSGSPRTSQQNVYNPS
EGSTWNPFDDDNFSKLTAEELLNKDFAKLGEGKHPEKLGGSAESLIPGFQSTQGDAFATTSFSAGTAEK
RKGGQTVDSGLPLLSVSDPFIPLQVPDAPEKLIEGLKSPDTSLLLPDLLPMTDPFGSTSDAVIEKADVA
VESLIPGLEPPVPQRLPSQTESVTSNRTDSLTGEDSLLDCSLLSNPTTDLLEEFAPTAISAPVHKAAED
SNLISGFDVPEGSDKVAEDEFDPIPVLITKNPQGGHSRNSSGSSESSLPNLARSLLLVDQLIDL.

Other isoforms of human AAK1 protein known in the art include those of SEQ ID NO: 2 and SEQ ID NO: 3:

(SEQ ID NO: 2)
MKKFFDSRREQGGSGLGSGSSGGGGSTSGLGSGYIGRVFGIGRQQVTVDEVLAEGGFAIVFLVRTSNGM
KCALKRMFVNNEHDLQVCKREIQIMRDLSGHKNIVGYIDSSINNVSSGDVWEVLILMDFCRGGQVVNLM
NQRLQTGFTENEVLQIFCDTCEAVARLHQCKTPIIHRDLKVENILLHDRGHYVLCDFGSATNKFQNPQT
EGVNAVEDEIKKYTTLSYRAPEMVNLYSGKIITTKADIWALGCLLYKLCYFTLPFGESQVAICDGNFTI
PDNSRYSQDMHCLIRYMLEPDPDKRPDIYQVSYFSFKLLKKECPIPNVQNSPIPAKLPEPVKASEAAAK
KTQPKARLTDPIPTTETSIAPRQRPKAGQTQPNPGILPIQPALTPRKRATVQPPPQAAGSSNQPGLLAS
VPQPKPQAPPSQPLPQTQAKQPQAPPTPQQTPSTQAQGLPAQAQATPQHQQQLFLKQQQQQQQPPPAQQ
QPAGTFYQQQQAQTQQFQAVHPATQKPAIAQFPVVSQGGSQQQLMQNFYQQQQQQQQQQQQQQLATALH
QQQLMTQQAALQQKPTMAAGQQPQPQPAAAPQPAPAQEPAIQAPVRQQPKVQTTPPPAVQGQKVGSLTP
PSSPKTQRAGHRRILSDVTHSAVFGVPASKSTQLLQAAAAEASLNKSKSATTTPSGSPRTSQQNVYNPS
EGSTWNPFDDDNESKLTAEELLNKDFAKLGEGKHPEKLGGSAESLIPGFQSTQGDAFATTSFSAGTAEK
RKGGQTVDSGLPLLSVSDPFIPLQVPDAPEKLIEGLKSPDTSLLLPDLLPMTDPFGSTSDAVIEKADVA
VESLIPGLEPPVPQRLPSQTESVTSNRTDSLTGEDSLLDCSLLSNPTTDLLEEFAPTAISAPVHKAAED
SNLISGFDVPEGSDKVAEDEFDPIPVLITKNPQGGHSRNSSGSSESSLPNLARSLLLVDQLIDL;
and
(SEQ ID NO: 3)
MKKFFDSRREQGGSGLGSGSSGGGGSTSGLGSGYIGRVFGIGRQQVTVDEVLAEGGFAIVFLVRTSNGM
KCALKRMFVNNEHDLQVCKREIQIMRDLSGHKNIVGYIDSSINNVSSGDVWEVLILMDFCRGGQVVNLM
NQRLQTGFTENEVLQIFCDTCEAVARLHQCKTPIIHRDLKVENILLHDRGHYVLCDFGSATNKFQNPQT
EGVNAVEDEIKKYTTLSYRAPEMVNLYSGKIITTKADIWALGCLLYKLCYFTLPFGESQVAICDGNFTI
PDNSRYSQDMHCLIRYMLEPDPDKRPDIYQVSYFSFKLLKKECPIPNVQNSPIPAKLPEPVKASEAAAK
KTQPKARLTDPIPTTETSIAPRQRPKAGQTQPNPGILPIQPALTPRKRATVQPPPQAAGSSNQPGLLAS
VPQPKPQAPPSQPLPQTQAKQPQAPPTPQQTPSTQAQGLPAQAQATPQHQQQLFLKQQQQQQQPPPAQQ
QPAGTFYQQQQAQTQQFQAVHPATQKPAIAQFPVVSQGGSQQQLMQNFYQQQQQQQQQQQQQQLATALH
QQQLMTQQAALQQKPTMAAGQQPQPQPAAAPQPAPAQEPAIQAPVRQQPKVQTTPPPAVQGQKVGSLTP
PSSPKTQRAGHRRILSDVTHSAVFGVPASKSTQLLQAAAAEASLNKSKSATTTPSGSPRTSQQNVYNPS
EGSTWNPFDDDNFSKLTAEELLNKDFAKLGEGKHPEKLGGSAESLIPGFQSTQGDAFATTSFSAGTAEK
RKGGQTVDSGLPLLSVSDPFIPLQVPDAPEKLIEGLKSPDTSLLLPDLLPMTDPFGSTSDAVIGKVIIS
VSSVMHDMCACFKNDKYLVNQSLGNSPATPEAKAI

In certain embodiments, the human BIKE/BMP2K protein has the following sequence of SEQ ID NO: 4 (although other isoforms are known in the art):

(SEQ ID NO: 4)
MKKFSRMPKSEGGSGGGAAGGGAGGAGAGAGCGSGGSSVGVRVFAVGRHQVTLEESLAEGGFSTVFLVR
THGGIRCALKRMYVNNMPDLNVCKREITIMKELSGHKNIVGYLDCAVNSISDNVWEVLILMEYCRAGQV
VNQMNKKLQTGFTEPEVLQIFCDTCEAVARLHQCKTPIIHRDLKVENILLNDGGNYVLCDFGSATNKFL
NPQKDGVNVVEEEIKKYTTLSYRAPEMINLYGGKPITTKADIWALGCLLYKLCFFTLPFGESQVAICDG
NFTIPDNSRYSRNIHCLIRFMLEPDPEHRPDIFQVSYFAFKFAKKDCPVSNINNSSIPSALPEPMTASE
AAARKSQIKARITDTIGPTETSIAPRQRPKANSATTATPSVLTIQSSATPVKVLAPGEFGNHRPKGALR
PGNGPEILLGQGPPQQPPQQHRVLQQLQQGDWRLQQLHLQHRHPHQQQQQQQQQQQQQQQQQQQQQQQQ
QQQHHHHHHHHLLQDAYMQQYQHATQQQQMLQQQFLMHSVYQPQPSASQYPTMMPQYQQAFFQQQMLAQ
HQPSQQQASPEYLTSPQEFSPALVSYTSSLPAQVGTIMDSSYSANRSVADKEAIANFTNQKNISNPPDM
SGWNPFGEDNFSKLTEEELLDREFDLLRSNRLEERASSDKNVDSLSAPHNHPPEDPFGSVPFISHSGSP
EKKAEHSSINQENGTANPIKNGKTSPASKDQRTGKKTSVQGQVQKGNDESESDFESDPPSPKSSEEEEQ
DDEEVLQGEQGDENDDDTEPENLGHRPLLMDSEDEEEEEKHSSDSDYEQAKAKYSDMSSVYRDRSGSGP
TQDLNTILLTSAQLSSDVAVETPKQEFDVFGAVPFFAVRAQQPQQEKNEKNLPQHRFPAAGLEQEEFDV
FTKAPFSKKVNVQECHAVGPEAHTIPGYPKSVDVFGSTPFQPFLTSTSKSESNEDLFGLVPFDEITGSQ
QQKVKQRSLQKLSSRQRRTKQDMSKSNGKRHHGTPTSTKKTLKPTYRTPERARRHKKVGRRDSQSSNEF
LTISDSKENISVALTDGKDRGNVLQPEESLLDPFGAKPFHSPDLSWHPPHQGLSDIRADHNTVLPGRPR
QNSLHGSFHSADVLKMDDFGAVPFTELVVOSITPHOSQQSQPVELDPFGAAPFPSKQ.

In certain embodiments, the human GAK protein has the following sequence of SEQ ID NO: 5 (although other isoforms are known in the art):

(SEQ ID NO: 5)
MSLLQSALDFLAGPGSLGGASGRDQSDFVGQTVELGELRLRVRRVLAEGGFAFVYEAQDVGSGREYALK
RLLSNEEEKNRAIIQEVCFMKKLSGHPNIVQFCSAASIGKEESDTGQAEFLLLTELCKGQLVEFLKKME
SRGPLSCDTVLKIFYQTCRAVQHMHRQKPPIIHRDLKVENLLLSNQGTIKLCDFGSATTISHYPDYSWS
AQRRALVEEEITRNTTPMYRTPEIIDLYSNFPIGEKQDIWALGCILYLLCFRQHPFEDGAKLRIVNGKY
SIPPHDTQYTVFHSLIRAMLQVNPEERLSIAEVVHQLQEIAAARNVNPKSPITELLEQNGGYGSATLSR
GPPPPVGPAGSGYSGGLALAEYDQPYGGFLDILRGGTERLFTNLKDTSSKVIQSVANYAKGDLDISYIT
SRIAVMSFPAEGVESALKNNIEDVRLFLDSKHPGHYAVYNLSPRTYRPSRFHNRVSECGWAARRAPHLH
TLYNICRNMHAWLRQDHKNVCVVHCMDGRAASAVAVCSFLCFCRLFSTAEAAVYMFSMKRCPPGIWPSH
KRYIEYMCDMVAEEPITPHSKPILVRAVVMTPVPLFSKQRSGCRPFCEVYVGDERVASTSQEYDKMRDF
KIEDGKAVIPLGVTVQGDVLIVIYHARSTLGGRLQAKMASMKMFQIQFHTGFVPRNATTVKFAKYDLDA
CDIQEKYPDLFQVNLEVEVEPRDRPSREAPPWENSSMRGLNPKILFSSREEQQDILSKFGKPELPRQPG
STAQYDAGAGSPEAEPTDSDSPPSSSADASRFLHTLDWQEEKEAETGAENASSKESESALMEDRDESEV
SDEGGSPISSEGQEPRADPEPPGLAAGLVQQDLVFEVETPAVLPEPVPQEDGVDLLGLHSEVGAGPAVP
PQACKAPSSNTDLLSCLLGPPEAASQGPPEDLLSEDPLLLASPAPPLSVQSTPRGGPPAAADPFGPLLP
SSGNNSQPCSNPDLFGEFLNSDSVTVPPSFPSAHSAPPPSCSADFLHLGDLPGEPSKMTASSSNPDLLG
GWAAWTETAASAVAPTPATEGPLFSPGGQPAPCGSQASWTKSQNPDPFADLGDLSSGLQGSPAGFPPGG
FIPKTATTPKGSSSWQTSRPPAQGASWPPQAKPPPKACTQPRPNYASNFSVIGAREERGVRAPSFAQKP
KVSENDFEDLLSNQGFSSRSDKKGPKTIAEMRKQDLAKDTDPLKLKLLDWIEGKERNIRALLSTLHTVL
WDGESRWTPVGMADLVAPEQVKKHYRRAVLAVHPDKAAGQPYEQHAKMIFMELNDAWSEFENQGSRPLF.

In certain embodiments, the human MPSK1 protein has the following sequence of SEQ ID NO: 6 (although other isoforms are known in the art):

(SEQ ID NO: 6)
MGHALCVCSRGTVIIDNKRYLFIQKLGEGGFSYVDLVEGLHDGHFYALKRILCHEQQDREEAQREADMH
RLFNHPNILRLVAYCLRERGAKHEAWLLLPFFKRGTLWNEIERLKDKGNFLTEDQILWLLLGICRGLEA
IHAKGYAHRDLKPTNILLGDEGQPVLMDLGSMNQACIHVEGSRQALTLQDWAAQRCTISYRAPELFSVQ
SHCVIDERTDVWSLGCVLYAMMFGEGPYDMVFQKGDSVALAVQNQLSIPQSPRHSSALRQLLNSMMTVD
PHQRPHIPLLLSQLEALQPPAPGQHTTQI.

In certain embodiments, methods disclosed herein are performed using an inhibitor of STK38 or STK38L. Serine/Threonine Kinase 38 (STK38) protein is a member of the AGC serine/threonine kinase family of proteins. The kinase activity of this protein is regulated by autophosphorylation and phosphorylation by other upstream kinases. This protein has been shown to function in the cell cycle and apoptosis. This protein has also been found to regulate the protein stability and transcriptional activity of the MYC oncogene. In addition, is has been shown that STK38 and STK38L phosphorylate AAK1. Alternative splicing results in multiple transcript variants of STK38. Serine/Threonine Kinase 38-like protein has been shown to enable ATP binding activity; magnesium ion binding activity; and protein serine/threonine kinase activity. It is involved in intracellular signal transduction and acts upstream of or within protein phosphorylation.

In certain embodiments, the human STK38 protein has the following sequence of SEQ ID NO: 7 (although other isoforms are known in the art):

(SEQ ID NO: 7)
MAMTGSTPCSSMSNHTKERVTMTKVTLENFYSNLIAQHEEREMRQKKLEKVMEEEGLKDEEKRLRRS
AHARKETEFLRLKRTRLGLEDFESLKVIGRGAFGEVRLVQKKDTGHVYAMKILRKADMLEKEQVGHI
RAERDILVEADSLWVVKMFYSFQDKLNLYLIMEFLPGGDMMTLLMKKDTLTEEETQFYIAETVLAID
SIHQLGFIHRDIKPDNLLLDSKGHVKLSDFGLCTGLKKAHRTEFYRNLNHSLPSDFTFQNMNSKRKA
ETWKRNRRQLAFSTVGTPDYIAPEVFMQTGYNKLCDWWSLGVIMYEMLIGYPPFCSETPQETYKKVM
NWKETLTFPPEVPISEKAKDLILRFCCEWEHRIGAPGVEEIKSNSFFEGVDWEHIRERPAAISIEIK
SIDDTSNFDEFPESDILKPTVATSNHPETDYKNKDWVFINYTYKRFEGLTARGAIPSYMKAAK.

In certain embodiments, the human STK38L protein has the following sequence of SEQ ID ON. 8 (although other isoforms are known in the art);

(SEQ ID NO: 8)
MAMTAGTTTTFPMSNHTRERVTVAKLTLENFYSNLILQHEERETRQKKLEVAMEEEGLADEEKKLRR
SQHARKETEFLRLKRTRLGLDDFESLKVIGRGAFGEVRLVQKKDTGHIYAMKILRKSDMLEKEQVAH
IRAERDILVEADGAWVVKMFYSFQDKRNLYLIMEFLPGGDMMTLLMKKDTLTEEETQFYISETVLAI
DAIHQLGFIHRDIKPDNLLLDAKGHVKLSDFGLCTGLKKAHRTEFYRNLTHNPPSDFSFQNMNSKRK
AETWKKNRRQLAYSTVGTPDYIAPEVEMQTGYNKLCDWWSLGVIMYEMLIGYPPFCSETPQETYRKV
MNWKETLVFPPEVPISEKAKDLILRFCIDSENRIGNSGVEEIKGHPFFEGVDWEHIRERPAAIPIEI
KSIDDTSNFDDFPESDILQPVPNTTEPDYKSKDWVFLNYTYKRFEGLTQRGSIPTYMKAGKL.

In certain embodiments, methods disclosed herein may be practiced with any agent capable of inhibiting expression or activity of a target gene, mRNA or protein, e.g., an inhibitor of a gene, mRNA or protein, complex or pathway disclosed herein, e.g., an inhibitor of an AAK1, GAK, or MPSK1 gene, mRNA, or protein. In particular embodiments, methods disclosed herein result in a decrease in an expression level or activity of a target gene, mRNA or protein, e.g., AAK1, in one or more cells or tissues (e.g., within a subject), e.g., as compared to the expression level or activity in control cells or tissue not contacted with the inhibitor, or a reference level, which may be pre-determined.

In particular embodiments, methods disclosed herein result in increased asymmetric cell division or increased cell polarity of one or more cell types, e.g., satellite cells, or in one or more tissues, e.g., skeletal muscle tissue (e.g., within a subject), e.g., as compared to the expression level or activity in control cells or tissue not contacted with the inhibitor, or a reference level.

Methods described herein may be practiced using any type of inhibitor that results in a reduced amount or level of a target gene, mRNA, or protein, e.g., in a cell or tissue, e.g., a cell or tissue in a subject. In particular embodiments, the inhibitor causes a reduction in active target protein, a reduction in total target protein, a reduction in target mRNA levels, and/or a reduction in target protein activity, e.g., in a cell or tissue contacted with the inhibitor. Methods of measuring total protein or mRNA levels, or activity, in a cell are known in the art. In certain embodiments, the inhibitor inhibits or reduces target protein activity or expression, e.g., mRNA and/or protein expression. In certain embodiments, the inhibitor causes increased degradation of the target protein, resulting in lower amounts of target protein in a cell or tissue.

Inhibitors refer to an agent capable of blocking or inhibiting expression and/or an activity of a target protein identified herein. Examples of various types of inhibitors include, but are not limited to, RNA interfering agents targeting target proteins, blocking antibodies against target proteins, small molecules and peptides that interfere with expression, function, or activity of any target protein. Another approach is the systemic or local delivery of a DNA plasmid encoding an inhibitor or a dominant negative form of a target protein. In addition, inhibitory agents include agents that inhibit or modulate expression, function, or activity of any target protein.

Inhibitors that may be used to practice the disclosed methods include but are not limited to agents that inhibit or reduce or decrease the expression or activity of a biomolecule, such as but not limited to a target gene, mRNA or protein, e.g., AAK1. In certain embodiments, an inhibitor can cause increased degradation of the biomolecule. In particular embodiments, an inhibitor can inhibit a biomolecule by competitive, uncompetitive, or non-competitive means. Exemplary inhibitors include, but are not limited to, nucleic acids, DNA, RNA, guide RNA (gRNA), short hairpin RNA (shRNA), short interfering RNA (siRNA), modified mRNA (mRNA), microRNA (miRNA), antisense RNA, proteins, protein mimetics, peptides, peptidomimetics, antibodies, small molecules, small organic molecules, inorganic molecules, chemicals, analogs that mimic the binding site of an enzyme, receptor, or other protein, e.g., that is involved in signal transduction, therapeutic agents, pharmaceutical compositions, drugs, and combinations of these. In some embodiments, the inhibitor can be a nucleic acid molecule including, but not limited to, siRNA, that reduces the amount of functional protein in a cell. Accordingly, compounds or agents said to be “capable of inhibiting” a particular target protein comprise any type of inhibitor.

In particular embodiments, an inhibitor comprises a nucleic acid that binds to a target gene or mRNA. Accordingly, a nucleic acid inhibitor may comprise a sequence complementary to a target polynucleotide sequence, or a region thereof, or an antisense thereof. In particular embodiments, a nucleic acid inhibitor comprises at least 8, at least 10, at least 12, at least 14, at least 16, at least 20, at least 24, or at least 30 nucleotide sequence corresponding to or complementary to a target polynucleotide sequence or antisense thereof. In various embodiments, a nucleic acid inhibitor comprises a region corresponding to or complementary to any nucleic acid target.

In certain embodiments, a target gene or mRNA is an Aakl (e.g., a human Aak1) gene or mRNA. In particular embodiments, the AAK1 is human AAK1 isoform 1, human AAK1 isoform 2, or human AAK1 isoform 3.

In certain embodiments, a target gene or mRNA is a GAK 1 (e.g., a human GAK) gene or mRNA.

In certain embodiments, a target gene or mRNA is a MPSK1 (e.g., a human MPSK1) gene or mRNA.

In certain embodiments, a nucleic acid inhibitor is an RNA interference or antisense RNA agent or a portion or mimetic thereof, or a morpholino, that decreases the expression of a target gene when administered to a cell. Typically, a nucleic acid inhibitor comprises at least a portion of a target nucleic acid molecule, or an ortholog thereof, or comprises at least a portion of the complementary strand of a target nucleic acid molecule. In some embodiments, expression of a target gene is reduced by at least about 10%, at least about 25%, at least about 50%, at least about 75%, or even 90-100%.

A “complementary” nucleic acid sequence is a nucleic acid sequence capable of hybridizing with another nucleic acid sequence comprised of complementary nucleotide base pairs. By “hybridize” is meant pair to form a double-stranded molecule between complementary nucleotide bases (e.g., adenine (A) forms a base pair with thymine (T), as does guanine (G) with cytosine (C) in DNA) under suitable conditions of stringency. (See, e.g., Wahl, G. M. and S. L. Berger (1987) Methods Enzymol. 152:399; Kimmel, A. R. (1987) Methods Enzymol. 152:507).

“Antisense” refers to a nucleic acid sequence, regardless of length, that is complementary to a nucleic acid sequence. In certain embodiments, antisense RNA refers to single stranded RNA molecules that can be introduced to an individual cell, tissue, or subject and results in decreased expression of a target gene through mechanisms that do not rely on endogenous gene silencing pathways. An antisense nucleic acid can contain a modified backbone, for example, phosphorothioate, phosphorodithioate, or others known in the art, or may contain non-natural internucleoside linkages. Antisense nucleic acid can comprise, e.g., locked nucleic acids (LNA).

“RNA interference” as used herein refers to the use of agents that decrease the expression of a target gene by degradation of a target mRNA through endogenous gene silencing pathways (e.g., Dicer and RNA-induced silencing complex (RISC)). RNA interference may be accomplished using various agents, including shRNA and siRNA. “Short hair-pin RNA” or “shRNA” refers to a double stranded, artificial RNA molecule with a hairpin turn that can be used to silence target gene expression via RNA interference (RNAi). Expression of shRNA in cells is typically accomplished by delivery of plasmids or through viral or bacterial vectors. shRNA is an advantageous mediator of RNAi in that it has a relatively low rate of degradation and turnover. Small interfering RNA (siRNA) is a class of double-stranded RNA molecules, usually 20-25 base pairs in length, similar to miRNA, and operating within the RNA interference (RNAi) pathway. It interferes with the expression of specific genes with complementary nucleotide sequences by degrading mRNA after transcription, preventing translation. In certain embodiments, an siRNA is 18, 19, 20, 21, 22, 23 or 24 nucleotides in length and has a 2 base overhang at its 3′ end. siRNAs can be introduced to an individual cell and/or culture system and result in the degradation of target mRNA sequences.

“Morpholino” as used herein refers to a modified nucleic acid oligomer wherein standard nucleic acid bases are bound to morpholine rings and are linked through phosphorodiamidate linkages. Similar to siRNA and shRNA, morpholinos bind to complementary mRNA sequences. However, morpholinos function through steric-inhibition of mRNA translation and alteration of mRNA splicing rather than targeting complementary mRNA sequences for degradation.

In certain embodiments, a nucleic acid inhibitor is a messenger RNA that may be introduced into a cell, wherein it encodes a polypeptide inhibitor of a target disclosed herein. In particular embodiments, the mRNA is modified, e.g., to increase its stability or reduce its immunogenicity, e.g., by the incorporation of one or more modified nucleosides. Suitable modifications are known in the art.

In certain embodiments, an inhibitor comprises an expression cassette that encodes a polynucleotide or polypeptide inhibitor of a target disclosed herein. In particular embodiments, the expression cassette is present in a gene therapy vector, for example a viral gene therapy vector. A variety of gene therapy vectors, including viral gene therapy vectors are known in the art, including, for example, AAV-based gene therapy vectors.

In some embodiments, an inhibitor is a polypeptide inhibitor. In particular embodiments, a polypeptide inhibitor binds to a target polypeptide, thus inhibiting its activity, e.g., kinase activity. Examples of polypeptide inhibitors include any types of polypeptides (e.g., peptides and proteins), such as antibodies and fragments thereof. In certain embodiments, an inhibitor is a dominant negative form of the target protein, e.g., a fragment of a target protein that binds a substrate but lacks enzymatic activity. In particular embodiments, the inhibitor is an antibody that binds a target protein, e.g., an antibody that inhibits activity of the target protein when bound to it. Antibodies that specifically bind to AAK1, GAK, or MPSK1 are available in the art and may be readily generated.

In certain embodiments, the inhibitor induces degradation of a target polypeptide. For example, inhibitors include proteolysis targeting chimeras (PROTAC), which induce selective intracellular proteolysis of target proteins. PROTACs include functional domains, which may be covalently linked protein-binding molecules: one is capable of engaging an E3 ubiquitin ligase, and the other binds to the target protein meant for degradation. Recruitment of the E3 ligase to the target protein results in ubiquitination and subsequent degradation of the target protein by the proteasome. In particular embodiments, an inhibitor is a PROTAC that targets any of the targets disclosed herein.

In particular embodiments, an inhibitor directly inhibits expression of or an activity of a target gene, mRNA, or protein, e.g., it may directly bind to the target gene, mRNA or protein. In some embodiments, the inhibitor indirectly inhibits expression of or an activity of a target gene, mRNA, or protein, e.g., it may bind to and inhibit a protein that mediates expression of the target gene, mRNA, or protein (such as a transcription factor), or it may bind to and inhibit another protein involved in activity of the target protein (such as another protein present in a complex with the target protein).

The effects/actions of inhibitors can be determined, e.g., by indicators of expression and/or one or more activity of a target protein in a cell.

In certain embodiments, the inhibitor comprises one or more components of a gene editing system. As used herein, the term “gene editing system” refers to a protein, nucleic acid, or combination thereof that is capable of modifying a target locus of an endogenous DNA sequence when introduced into a cell. Numerous gene editing systems suitable for use in the methods of the present invention are known in the art including, but not limited to, zinc-finger nuclease systems, transcription activator-like effector nucleases (TALEN) systems, meganucleases or argonaute-based systems (Nat Biotechnol. 2016 July; 34 (7): 768-73) or base editors (Komor et al., Nature 533, 420-424, doi: 10.1038/nature17946), and CRISPR/Cas systems. The disclosure encompasses the use of any of these alternative means for site specific DNA editing, e.g., introducing an inhibitory mutation into a target gene, e.g., Aak1.

In some embodiments, the gene editing system used in the methods described herein is a CRISPR (Clustered Regularly Interspaced Short Palindromic Repeats)/Cas (CRISPR Associated) nuclease system, which is an engineered nuclease system based on a bacterial system that can be used for mammalian genome engineering. Generally, the system comprises a CRISPR-associated endonuclease (for example, a Cas endonuclease) and a guide RNA (gRNA). The gRNA is comprised of two parts; a crispr-RNA (crRNA) that is specific for a target genomic DNA sequence, and a trans-activating RNA (tracrRNA) that facilitates endonuclease binding to the DNA at the targeted insertion site. In some embodiments, the crRNA and tracrRNA may be present in the same RNA oligonucleotide, referred to as a single guide-RNA (sgRNA). In some embodiments, the crRNA and tracrRNA may be present as separate RNA oligonucleotides. In such embodiments, the gRNA is comprised of a crRNA oligonucleotide and a tracrRNA oligonucleotide that associate to form a crRNA: tracrRNA duplex. As used herein, the term “guide RNA” or “gRNA” refers to the combination of a tracrRNA and a crRNA, present as either an sgRNA or a crRNA: tracrRNA duplex.

In some embodiments, the CRISPR/Cas systems comprise a Cas protein, a crRNA, and a tracrRNA. In some embodiments, the crRNA and tracrRNA are combined as a duplex RNA molecule to form a gRNA. In some embodiments, the crRNA: tracrRNA duplex is formed in vitro prior to introduction to a cell. In some embodiments, the crRNA and tracrRNA are introduced into a cell as separate RNA molecules and crRNA: tracrRNA duplex is then formed intracellularly. In some embodiments, polynucleotides encoding the crRNA and tracrRNA are provided. In such embodiments, the polynucleotides encoding the crRNA and tracrRNA are introduced into a cell and the crRNA and tracrRNA molecules are then transcribed intracellularly. In some embodiments, the crRNA and tracrRNA are encoded by a single polynucleotides. In some embodiments, the crRNA and tracrRNA are encoded by separate polynucleotides.

In some embodiments, a Cas endonuclease is directed to the target insertion site by the sequence specificity of the crRNA portion of the gRNA, which may include a protospacer motif (PAM) sequence near the target insertion site. A variety of PAM sequences suitable for use with a particular endonuclease (e.g., a Cas9 endonuclease) are known in the art (See e.g., Nat Methods. 2013 November; 10 (11): 1116-1121 and Sci Rep. 2014; 4:5405).

The specificity of a gRNA for a target locus is mediated by the crRNA sequence, which comprises a sequence of about 20 nucleotides that are complementary to the DNA sequence at a target locus, e.g., complementary to a target DNA sequence. In some embodiments, the crRNA sequences used in the methods of the present invention are at least 90% complementary to a DNA sequence of a target locus. In some embodiments, the crRNA sequences used in the methods of the present invention are at least 95%, 96%, 97%, 98%, or 99% complementary to a DNA sequence of a target locus. In some embodiments, the crRNA sequences used in the methods of the present invention are 100% complementary to a DNA sequence of a target locus. In some embodiments, the crRNA sequences described herein are designed to minimize off-target binding using algorithms known in the art (e.g., Cas-OFF finder) to identify target sequences that are unique to a particular target locus or target gene.

In some embodiments, the endonuclease is a Cas protein or ortholog. In some embodiments, the endonuclease is a Cas9 protein. In some embodiments, the Cas9 protein is derived from Streptococcus pyogenes (e.g., SpCas9), Staphylococcus aureus (e.g., SaCas9), or Neisseria meningitides (NmeCas9). In some embodiments, the Cas endonuclease is a Cas9 protein or a Cas9 ortholog and is selected from the group consisting of SpCas9, SpCas9-HF1, SpCas9-HF2, SpCas9-HF3, SpCas9-HF4, SaCas9, FnCpf, FnCas9, eSpCas9, and NmeCas9. In some embodiments, the endonuclease is selected from the group consisting of C2C1, C2C3, Cpf1 (also referred to as Cas12a), Casl, CaslB, Cas2, Cas3, Cas4, Cas5, Cas6, Cas7, Cas8, Cas9 (also known as Csn1 and Csx12), Cas10, Csy1, Csy2, Csy3, Cse1, Cse2, Cscl, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4. In some embodiments, the Cas9 is a Cas9 nickase mutant. Cas9 nickase mutants comprise only one catalytically active domain (either the HNH domain or the RuvC domain).

In certain embodiments, an inhibitor is a small molecule inhibitor, or a stereoisomer, enantiomer, diastereomer, isotopically-enriched, pro-drug, or pharmaceutically acceptable salt thereof. A variety of small molecule inhibitors of targets disclosed herein, including AAK1, are known and available.

In particular embodiments, an AAK1 inhibitor inhibits or reduces AAK1 enzymatic activity, e.g., phosphorylation the AP2M1/mu2 subunit of the adaptor protein complex 2 (AP-2) and/or NUMB.

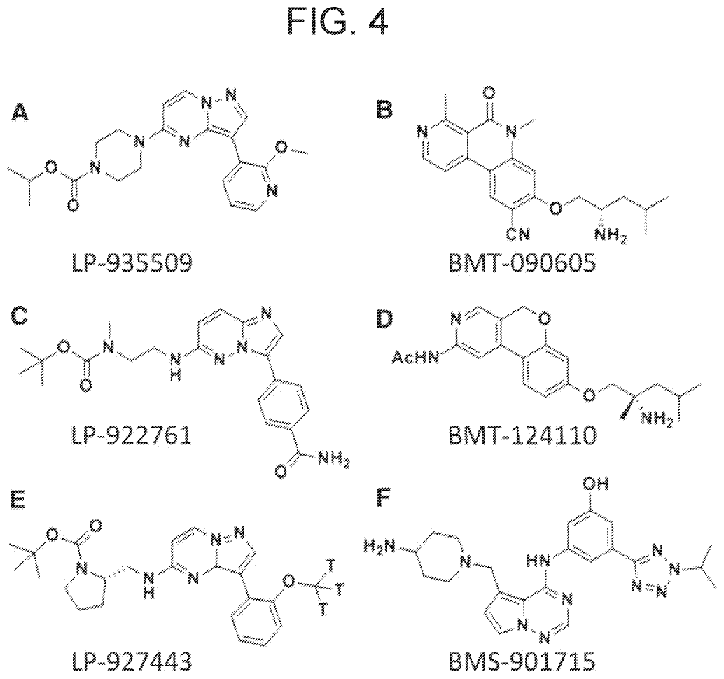

In particular embodiments, AAK1 inhibitors include, but are not limited to: LX9211, SGC-AAK1-1, LP-935509, LP-922761, BMT-090605, BMT-124110, LP-927443, and BMS-901715. The structures of illustrative AAK1 inhibitors are shown in FIG. 4.

LX9211 is described in PCT Application Publication No. WO2015153720. LX9211 is used interchangeably with SAT3003 herein. AAK1 inhibitors include those disclosed in this application, including(S)-1-((2′,6-bis(difluoromethyl)-[2,4′-bipyridin]-5-yl)oxy)-2,4-dimethylpentan-2-amine and pharmaceutically acceptable salts and solvates thereof, including LX9211 and salts thereof:

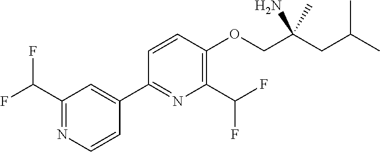 [SMILES: CC(C)C[C@](C)(N)COc1ccc(-c2ccnc(C(F)F)c2)nc1C(F)F]

SGC-AAK1-1 (N-(6-(3-(N,N-Diethylsulfamoylamino)phenyl)-1H-indazol-3-yl) cyclopropanecarboxamide) is an ATP-competitive kinase inhibitor against AP2-associated protein kinase 1/AAK1 and BMP-2-inducible protein kinase/BIKE/BMP2K (Ki=9.1 & 17 nM, respectively, by ATP site fluorescent tracer displacement assay; AAK1 IC50=270 nM by coupled enzyme assay). SGC-AAK1-1 downregulates cellular AP2M1 Thr156 phosphorylation level in a dose-dependent manner (ECmax ˜12.5 μM) without detectable cytotoxicity.

LP-935509 is a selective, ATP-competitive and brain-penetrant inhibitor of adapter protein-2 associated kinase 1 (AAK1) with an IC50 and a Ki of 3.3 nM and 0.9 nM, respectively.

LP-922761 is a potent, selective and orally active adapter protein-2 associated kinase 1 (AAK1) inhibitor with IC₅₀s of 4.8 nM and 7.6 nM in enzyme and cell assays, respectively. LP-922761 also inhibits BMP-2-inducible protein kinase (BIKE) with an IC₅₀ of 24 nM.

BMT-090605 is a potent, selective AAK1 inhibitor, with an IC₅₀ of 0.6 nM. BMT-090605 shows antinociceptive activity. BMT-090605 inhibits BMP-2-inducible protein kinase (BIKE) with an IC₅₀ of 45 nM.

BMT-124110 is a potent, selective AAK1 inhibitor with an IC₅₀ of 0.9 nM. BMT-124110 shows antinociceptive activity. BMT-090605 inhibits BMP-2-inducible protein kinase (BIKE) with an IC₅₀ of 17 nM.

BMS-901715 is a potent, selective adapter protein-2 associated kinase 1 (AAK1) inhibitor.

Additional AAK1 inhibitors that may be used according to the disclosure include, but are not limited to, those described in the following references: Hartz, R. A. et al. Discovery, Structure—Activity Relationships, and In Vivo Evaluation of Novel Aryl Amides as Brain Penetrant Adaptor Protein 2-Associated Kinase 1 (AAK1) Inhibitors for the Treatment of Neuropathic Pain. J Med Chem 64, 11090-11128 (2021); Verdonck, S. et al. Synthesis and Structure-Activity Relationships of 3,5-Disubstituted-pyrrolo[2,3-b]pyridines as Inhibitors of Adaptor-Associated Kinase 1 with Antiviral Activity. J Med Chem 62, 5810-5831 (2019); Wells, C. et al. SGC-AAK1-1: A Chemical Probe Targeting AAK1 and BMP2K. Acs Med Chem Lett 11, 340-345 (2020); Kostich, W. et al. Inhibition of AAK1 Kinase as a Novel Therapeutic Approach to Treat Neuropathic Pain. J Pharmacol Exp Ther 358, 371-386 (2016); Hesselink. LX9211 A Selective Inhibitor of AAK1 (Adapter-Associated Kinase) for Neuropathic Pain? Some Thoughts on Selectivity and Specificity. Austin Neurology 3, 1013-(2018); Martinez-Gualda, B., Schols, D. & Jonghe, S. D. A patent review of adaptor associated kinase 1 (AAK1) inhibitors (2013-present). Expert Opin Ther Pat 31, 1-26 (2021). Additional AAK1 inhibitors include, but are not limited to: 3-methyloxetan-3-yl-4-(3-(2-methoxypyridin-3-yl) pyrazolo[1,5-a]pyrimidin-5-yl) piperazine-1-carboxylate, and pharmaceutically acceptable salts thereof (described in U.S. Patent Publication No. 20160039824).

Other examples of AAK1 inhibitors include, but are not limited to those disclosed in PCT Application Publication Nos. WO2013134219 (2013), WO2015035167 (2015), WO2015026574 (2015), WO2013134336 (2013), WO2013134228 (2013), WO2015142714 (2015), WO2015035117 (2015), WO2015153720 (2015), WO2017059085 (2017), WO2015006100 (2015), WO2013134036 (2013), WO2015038112 (2015), WO2014130258 (2014), WO2016164295 (2016), WO2014022167 (2014), WO2015116060 (2015), WO2015054358 (2015), WO2015116492 (2015), WO2016022312 (2016), and WO2015142714.

In particular embodiments, GAK (cyclin G-associated kinase) inhibitors include, but are not limited to: SGC-AAK1-1 and other described in Wells, C. et al. SGC-AAK1-1: A Chemical Probe Targeting AAK1 and BMP2K. Acs Med Chem Lett 11, 340-345 (2020).

In particular embodiments, MPSK1 (myristoylated and palmitoylated serine/threonine kinase 1) inhibitors include, but are not limited to: SGC-AAK1-1 and other described in Wells, C. et al. SGC-AAK1-1: A Chemical Probe Targeting AAK1 and BMP2K. Acs Med Chem Lett 11, 340-345 (2020).

### Pharmaceutical Compositions

In particular aspects, the disclosure includes compositions, e.g., pharmaceutical compositions, comprising an inhibitor disclosed herein, e.g., an AAK1 inhibitor, including any of the various classes of inhibitors described herein. The invention encompasses pharmaceutical compositions comprising an inhibitor and a pharmaceutically acceptable carrier, diluent or excipient. Any inert excipient that is commonly used as a carrier or diluent may be used in compositions of the present invention, such as sugars, polyalcohols, soluble polymers, salts and lipids. Sugars and polyalcohols which may be employed include, without limitation, lactose, sucrose, mannitol, and sorbitol. Illustrative of the soluble polymers which may be employed are polyoxyethylene, poloxamers, polyvinylpyrrolidone, and dextran. Useful salts include, without limitation, sodium chloride, magnesium chloride, and calcium chloride. Lipids which may be employed include, without limitation, fatty acids, glycerol fatty acid esters, glycolipids, and phospholipids.

In addition, the pharmaceutical compositions may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate, Primogel), buffers (e.g., tris-HCL, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol, cyclodextrins), a glidant (e.g., colloidal silicon dioxide), anti-oxidants (e.g., ascorbic acid, sodium metabisulfite, butylated hydroxyanisole), stabilizers (e.g., hydroxypropyl cellulose, hydroxypropylmethyl cellulose), viscosity increasing agents (e.g., carbomer, colloidal silicon dioxide, ethyl cellulose, guar gum), sweeteners (e.g., sucrose, aspartame, citric acid), flavoring agents (e.g., peppermint, methyl salicylate, or orange flavoring), preservatives (e.g., thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate, methyl cellulose, hydroxyethyl cellulose, carboxymethylcellulose sodium), polymer coatings (e.g., poloxamers or poloxamines), coating and film forming agents (e.g., ethyl cellulose, acrylates, polymethacrylates) and/or adjuvants.

In one embodiment, the pharmaceutical compositions are prepared with carriers that will protect the inhibitor against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Additionally, the invention encompasses pharmaceutical compositions comprising any solid or liquid physical form of an inhibitor. For example, the inhibitor can be in a crystalline form, in amorphous form, and have any particle size. The particles may be micronized, or may be agglomerated, particulate granules, powders, oils, oily suspensions or any other form of solid or liquid physical form.

When inhibitors exhibit insufficient solubility, methods for solubilizing the compounds may be used. Such methods are known to those of skill in this art, and include, but are not limited to, pH adjustment and salt formation, using co-solvents, such as ethanol, propylene glycol, polyethylene glycol (PEG) 300, PEG 400, DMA (10-30%), DMSO (10-20%), NMP (10-20%), using surfactants, such as polysorbate 80, polysorbate 20 (1-10%), cremophor EL, Cremophor RH40, Cremophor RH60 (5-10%), Pluronic F68/Poloxamer 188 (20-50%), Solutol HS15 (20-50%), Vitamin E TPGS, and d-a-tocopheryl PEG 1000 succinate (20-50%), using complexation such as HP β-CD and SBE β-CD (10-40%), and using advanced approaches such as micelles, addition of a polymer, nanoparticle suspensions, and liposome formation.

Inhibitors may also be administered or coadministered in slow release dosage forms. Inhibitors may be in gaseous, liquid, semi-liquid or solid form, formulated in a manner suitable for the route of administration to be used. For oral administration, suitable solid oral formulations include tablets, capsules, pills, granules, pellets, sachets and effervescent, powders, and the like. Suitable liquid oral formulations include solutions, suspensions, dispersions, syrups, emulsions, oils and the like. For parenteral administration, reconstitution of a lyophilized powder is typically used.

Suitable doses of the inhibitors for use in treating the diseases or disorders described herein can be determined by those skilled in the relevant art. Therapeutic doses are generally identified through a dose ranging study in humans based on preliminary evidence derived from the animal studies. Doses should be sufficient to result in a desired therapeutic benefit without causing unwanted side effects. Mode of administration, dosage forms and suitable pharmaceutical excipients can also be well used and adjusted by those skilled in the art. All changes and modifications are envisioned within the scope of the present patent application.

In certain embodiments, the disclosure includes unit dosage forms of a pharmaceutical composition comprising an agent that inhibits expression or activity of a target polypeptide (or results in reduced levels of a target protein) and a pharmaceutically acceptable carrier, diluent or excipient, wherein the unit dosage form is effective to increase expression of a hemoglobin gamma in one or more tissue in a subject to whom the unit dosage form is administered.

In particular embodiments, the unit dosage forms comprise an effective amount, an effective concentration, and/or an inhibitory concentration, of an inhibitor to treat a disease or disorder disclosed herein, e.g., DMD.

### EXAMPLES

### Example 1: AAK1 Inhibition Enhances Asymmetric Division of Satellite Cells

The effect of AAK1 inhibition on asymmetric division of satellite cells was first examined by contacting cultured muscle fibers with a small interfering RNA (siRNA) specific for AAK1.

Myf5-Cre/R26R-eYFP transgenic mice, possessing a knock-in of Cre recombinase in the coding-region of the myogenic commitment factor Myf5 (Tallquist et al. Development, 2000. 127 (23): p. 5059-70), crossed with the knock-in of Cre-activated yellow fluorescent protein (eYFP) at the ROSA26 locus (Srinivas et al. BMC Dev Biol, 2001. 1: p. 4), were used as a lineage reporter model to discriminate committed satellite myogenic cells that have expressed Myf5-Cre (eYFPPos) from satellite stem cells that have never expressed Myf5-Cre (eYFPNeg) (Kuang et al. 2007). Myf5-Cre/R26R-nTnG transgenic mice possess a CMV/β-actin promoter, a loxP flanked nuclear TdTomato (nTdT) and nuclear GFP (nGFP) cassette within the ROSA.26 (Sor) locus. When crossed with the Myf5-Cre transgenic line, all cells express nTdT except those cells that have expressed Myf5, which express a nGFP signal. These transgenic models allow for the visualization and enumeration of de novo Myf5 expression in committed daughter cells during asymmetric divisions (Kuang et al. 2007; Wang et al. Cell Stem Cell, 2019. 24 (3): p. 419-432 e6; Le Grand et al. Cell Stem Cell, 2009. 4 (6): p. 535-47).

Myofiber culture was performed as described previously (Dumont et al. Nat Med, 2015. 21 (12): p. 1455-63). Briefly, extensor digitorum longus muscles (EDL) were carefully dissected and incubated at 37° C. in DMEM with 2% L-glutamine, 4.5% glucose, and 110 mg/mL sodium pyruvate (Gibco) containing 0.2% collagenase I (Sigma) for 45 min. Myofibers were isolated using gentle trituration in DMEM+ with 2% L-glutamine, 4.5% glucose, and 110 mg/ml sodium pyruvate (Gibco) with a glass pipet. Myofibers were cultured at 37° C. for 42, or 72 h in DMEM+ with 2% L-glutamine, 4.5% glucose, and 110 mg/ml sodium pyruvate (Gibco) containing 20% FBS (Wisent), 1% chick embryo extract (MP Biomedicals), and 2.5 ng/ml bFGF (Cedarlane). Transfection of satellite cells on myofibers was performed using lipofectamine RNAimax (Life Technologies) and validated Smartpool siRNAs for AAK1 or scramble (SCR) (Dharmacon) at a final concentration of 50 nM. To ensure maximal efficiency, two transfections were performed at 4 h and 16 h after isolation of the myofibers as described previously (Wang et al. Cell Stem Cell, 2019. 24 (3): p. 419-432 e6). A pool of 4 siRNAs targeting AAK1 was used. The siRNA sequences were as follows: GAAGGUGGAUUCGCUCUUG (SEQ ID NO: 9); GGACUCAAAUCUCCUGACA (SEQ ID NO: 10); GCAGAUAUUUGGGCUCUAG (SEQ ID NO: 11); and AAAUGUGCCUUGAAACGUA (SEQ ID NO: 12).

The % of myogenin (MyoG+) cells, total number (#) of satellite cells per fiber, #of myogenic cells per fiber, % of YFP-satellite stem cells, #of symmetric satellite stem cell divisions, #of asymmetric satellite stem cell divisions, proportion of asymmetric stem cell divisions, rate of YFP-divisions, and rate of YFP+ divisions were quantified by enumerating satellite cells and their progeny in the cultured myofibers. Enumeration was performed via immunocytochemistry, by immunostaining cells on the myofibers with anti-Pax7 antibodies (42 h) or anti-Pax7 antibodies and anti-Myogenin antibodies (72 h) and counting them manually in a blinded manner.

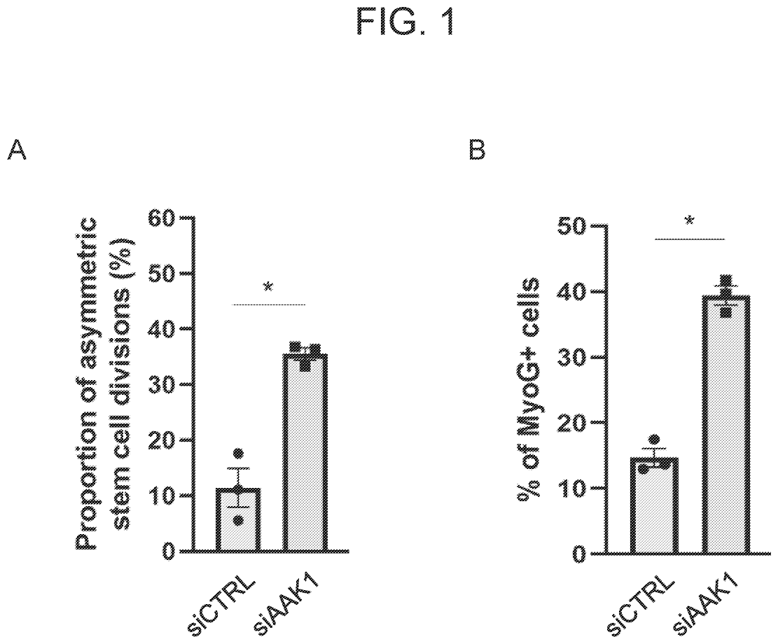

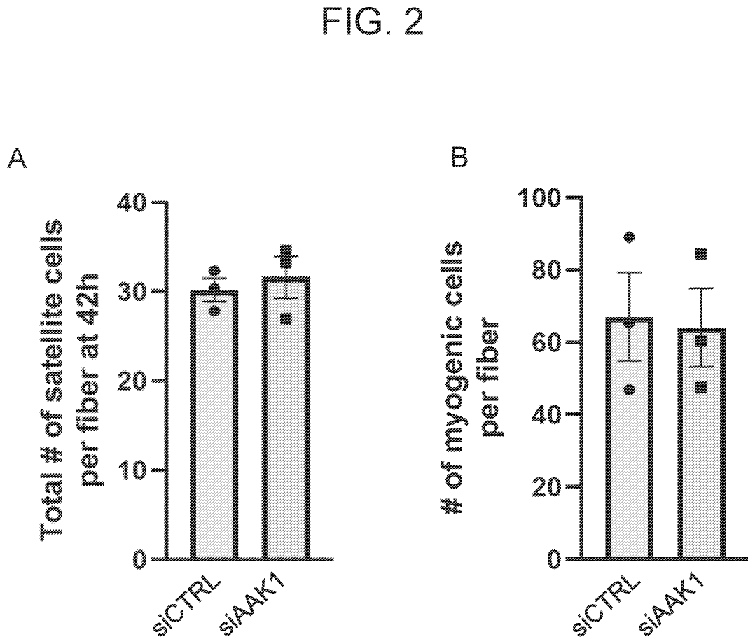

AAK1 inhibition by siRNA resulted in a ˜3-fold increase in the proportion of asymmetric satellite stem cell divisions after 42 h in culture (FIG. 1A), and the proportion of committed myogenic cells after 72 h in culture (FIG. 1B). Notably, there was also no change in the total number of satellite cells at 42 h in culture (FIG. 2A), or total number of myogenic cells at 72 h in culture (FIG. 2B), suggesting AAK1 inhibition did not have an effect on cell cycle progression.

The effect of siRNA inhibition of other genes, including Gak, Bmp2k, MPSK1, and Numb, was also examined as described above, and compared to siRNA inhibition of Aak1 and/or control siRNA. The sequences of the pool of 4 siRNAs targeting BMP2K/BIKE included: GCAGGUAUCACCCGAGUAU (SEQ ID NO: 13), UAUCCUACUUUGCGUUUAA (SEQ ID NO: 14), GGGAAGUGCUUAUCUUAAU (SEQ ID NO: 15), and GCUCAAGUCCACUAUGUAA (SEQ ID NO: 16). The sequences of the pool of 4 siRNAs targeting GAK included: GAGGGAGGCUGCAGGCUAA (SEQ ID NO: 17), GACCAAACAGCAAGACUUA (SEQ ID NO: 18), UGGCAGAGAGUAUGCAUUA (SEQ ID NO: 19), and CCUGGAUGCUUGUGAUAUU (SEQ ID NO: 20). The sequences of the pool of 4 siRNAs targeting MPSK1 included: GAAAGAACGAGGUGCUAAG (SEQ ID NO: 21), UCAGUCAGUUGGAGGCAUU (SEQ ID NO: 22), ACCCAAAUCUGAUCAAAUC (SEQ ID NO: 23), and GGACUUGGGUUCUAUGAAU (SEQ ID NO: 24).

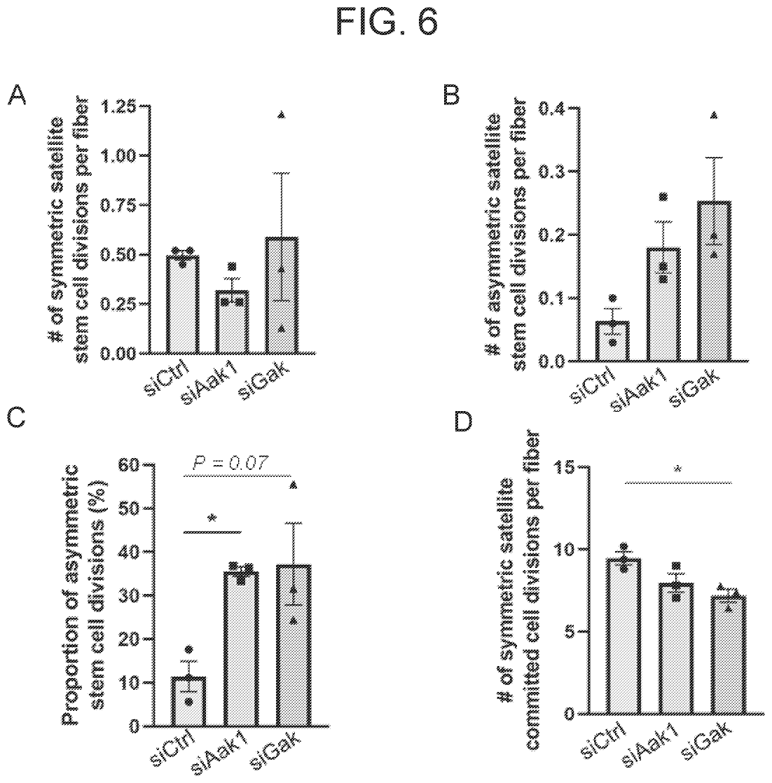

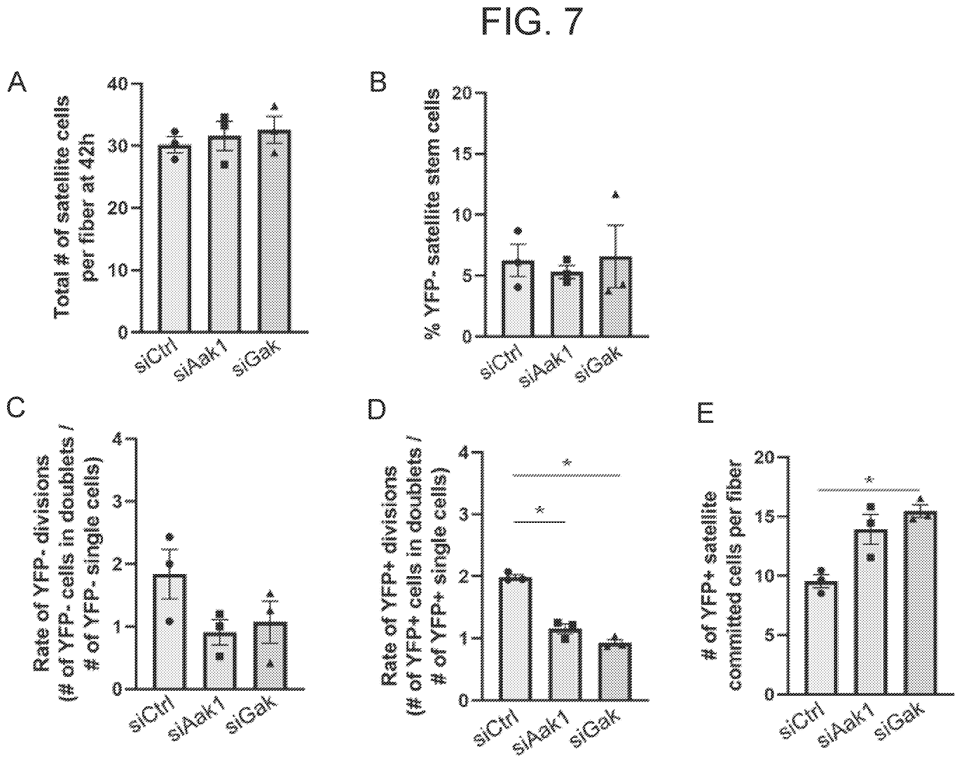

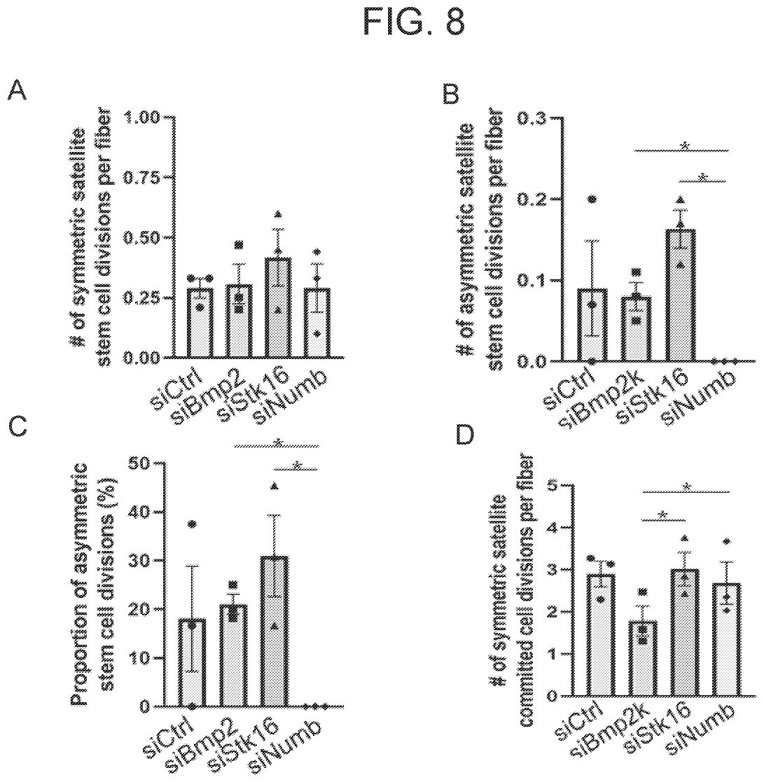

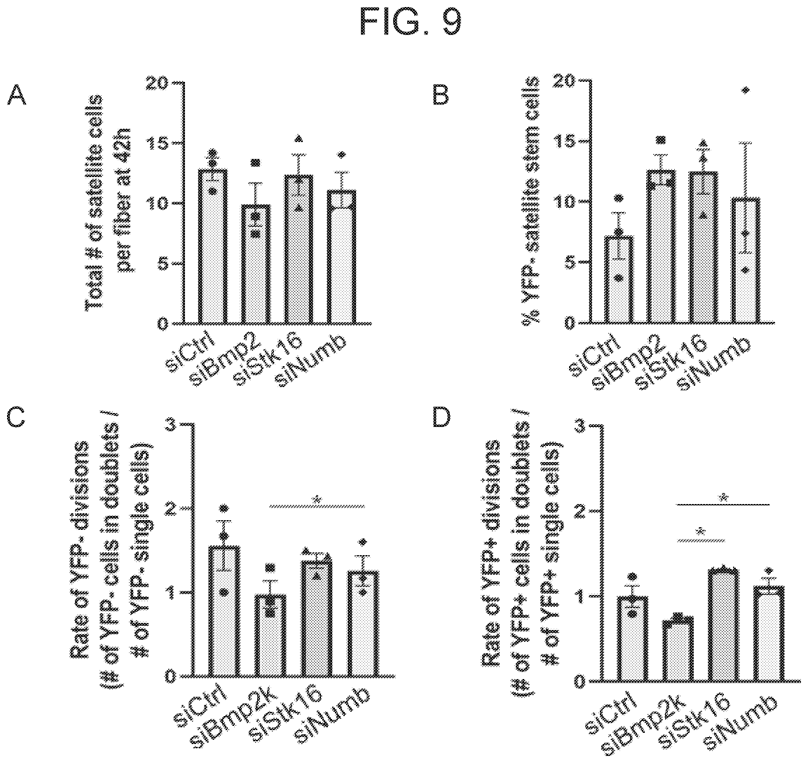

siRNA inhibition of Gak had a similar effect as siRNA inhibition of Aak1, resulting in an increased number and proportion of asymmetric satellite cell divisions (FIGS. 6B and 6C), a reduced rate of YFP− and YFP+ divisions (FIGS. 7C and 7D), and an increased number of YFP+ satellite committed cells per fiber (FIG. 7E). siRNA inhibition of MPSK1 also increased the number and proportion of asymmetric satellite cell divisions (FIGS. 8B and 8C). However, siRNA inhibition of Bmp2k had no significant effect on the number or proportion of asymmetric satellite cell divisions, whereas inhibition of Numb resulted in a complete loss of asymmetric divisions (FIGS. 8B and 8C).

The effect of pharmacological inhibition of AAK1 on asymmetric division of satellite cells was also examined by contacting satellite cells cultured on isolated myofibers with small molecule inhibitors of AAK1. Either SGC-AAK1-1 (Cat. No. HY-117626, MedChemExpress) or LP-935509 (Cat. No. HY-123940, MedChemExpress) was added to the culture medium at a final concentration of 100 nM, and equal dilution of DMSO was used as vehicle control.

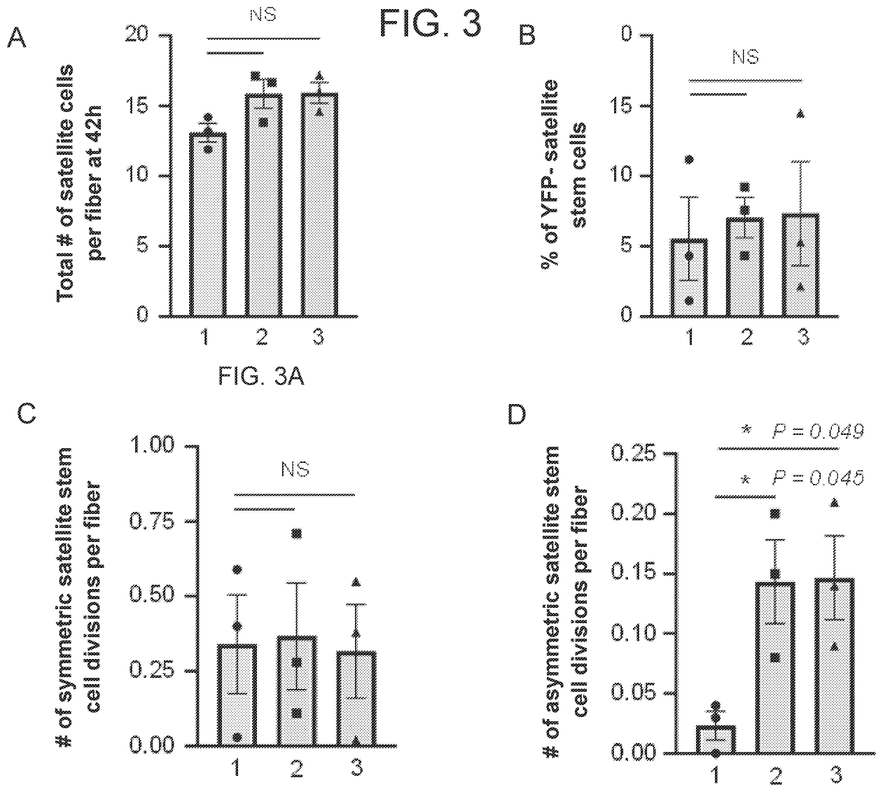

Pharmacological inhibition of AAK1 resulted in no change in the total number of Pax7-expressing satellite cells (FIG. 3A), no change in the total number of YFP-negative satellite stem cells (FIG. 3B), and no change in the number of symmetric satellite stem cell divisions (FIG. 3C). However, treatment with the AAK1 inhibitors caused a significant 6-fold increase in the number of asymmetric satellite stem cells divisions (FIG. 3D).

These results demonstrate that AAK1 inhibition enhances asymmetric satellite cell division and support the use of AAK1 inhibition to treat diseases and injuries that would benefit from increased asymmetric satellite cell division and tissue regeneration.

### Example 2: AAK1 Inhibition Stimulates Asymmetric Division of Dystrophin-Deficient MDX Muscle Stem Cells

The effect of AAK1 inhibition on asymmetric division of satellite cells was examined using tissue obtained from Mdx mice, essentially as described in Example 1. Mdx mouse are an established model of Duchenne muscular dystrophy (see, e.g., Swiderski, K. and Lynch, G. S., Am J Phsiol Cell Physiol 2021 Aug. 1; 321 (2): C409-C412, EPub 2021 Jul. 14).

Satellite cells cultured on isolated myofibers were contacted with small molecule inhibitors of AAK1. Either SGC-AAK1-1 (Cat. No. HY-117626, MedChemExpress) or LP-935509 (Cat. No. HY-123940, MedChemExpress) was added to the culture medium at a final concentration of 100 nM, and equal dilution of DMSO was used as vehicle control.

The total number (#) of satellite cells per fiber, % of YFP-satellite stem cells, number of symmetric satellite stem cell divisions, and proportion of asymmetric stem cell divisions were quantified by enumerating satellite cells and their progeny in the cultured myofibers. Enumeration was performed via immunocytochemistry, by immunostaining cells on the myofibers with anti-Pax7 antibodies (42 h) or anti-Pax7 antibodies and anti-Myogenin antibodies (72 h) and counting them manually in a blinded manner.

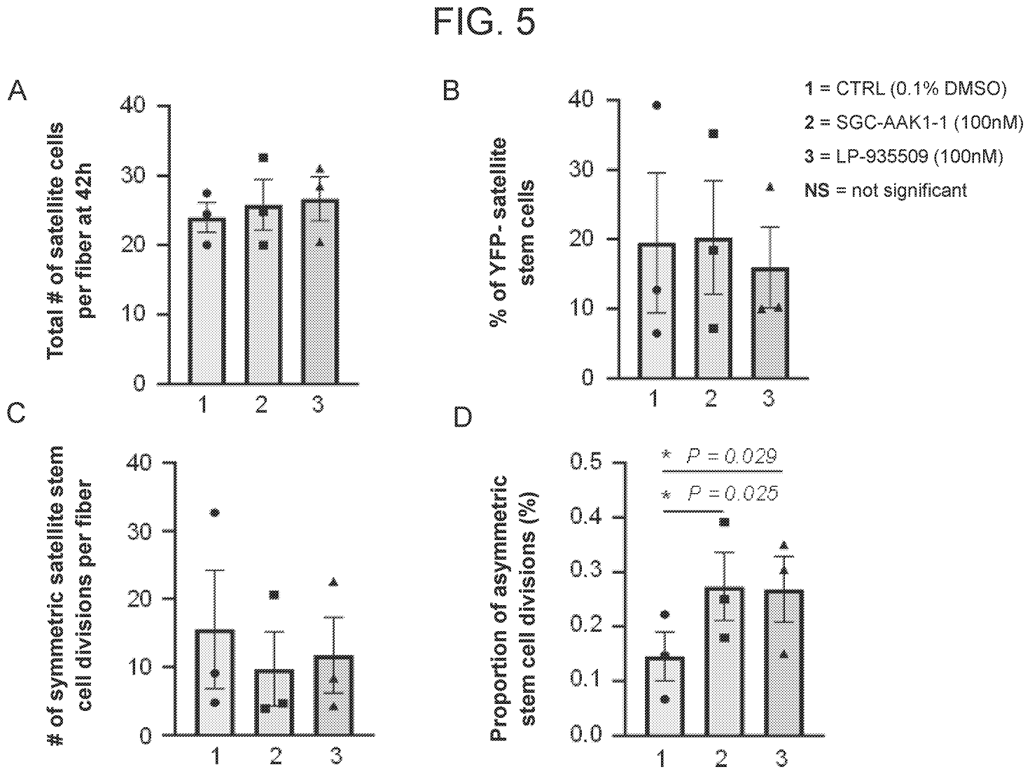

Pharmacological inhibition of AAK1 resulted in no change in the total number of Pax7-expressing satellite cells (FIG. 5A), no change in the total number of YFP-negative satellite stem cells (FIG. 5B), and no change in the number of symmetric satellite stem cell divisions (FIG. 5C). However, treatment with the AAK1 inhibitors caused a significant increase in the number of asymmetric satellite stem cell divisions (FIG. 5D).

These data support a functional rescue of skeletal muscle satellite cells in Duchenne muscular dystrophy (DMD) and other muscle diseases, and enhancement of muscle regeneration via AAK1 inhibition, by restoring the balance between self-renewal of satellite cells and expansion of myogenic progenitor cells.

### Example 3: Inhibition of AAK1 In Vivo Increases Myogenic Progenitor Density and Myofiber Feret

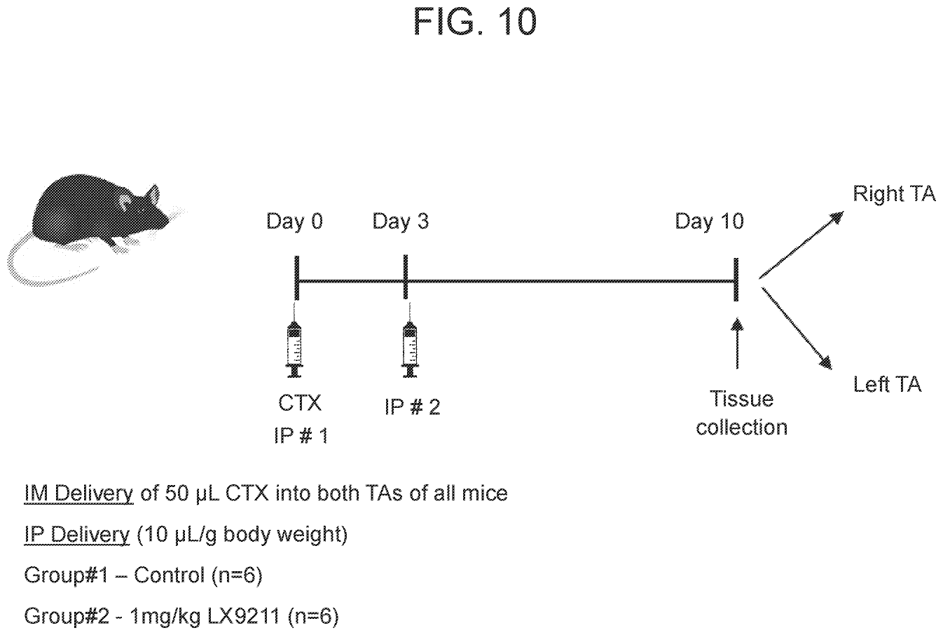

Because LX9211 (SAT-3003)-mediated inhibition of AAK1 stimulated the asymmetric division of mdx satellite stem cells, the effect of LX9211 was next examined in vivo, using a C57BL/10ScSn-Dmd^{mdx}/J (mdx) mouse model of Duchenne muscular dystrophy. LX9211 (1 mg/kg) or DMSO (control) was administered (intraperitoneally, 10 μl/g body weight) concurrently (day 0) with intramuscular injection of cardiotoxin (CDX, intramuscularly, 50 μl into both tibialis anterior [TA] muscles) and again on day 3. Ten days after CDX injection, the left TA was harvested for a single cell assay: it was processed by enzymatic digestion for mononuclear cell isolation, then filtered, plated and stained for Pax7 and Myogenin (MyoG) using anti-Pax7 antibodies and anti-MyoG antibodies prior to imaging. The right TA was harvested for histology: it was cryosectioned, then immunostained for Pax7 and MyoG, prior to imaging via high throughput Opera analysis (FIG. 10).

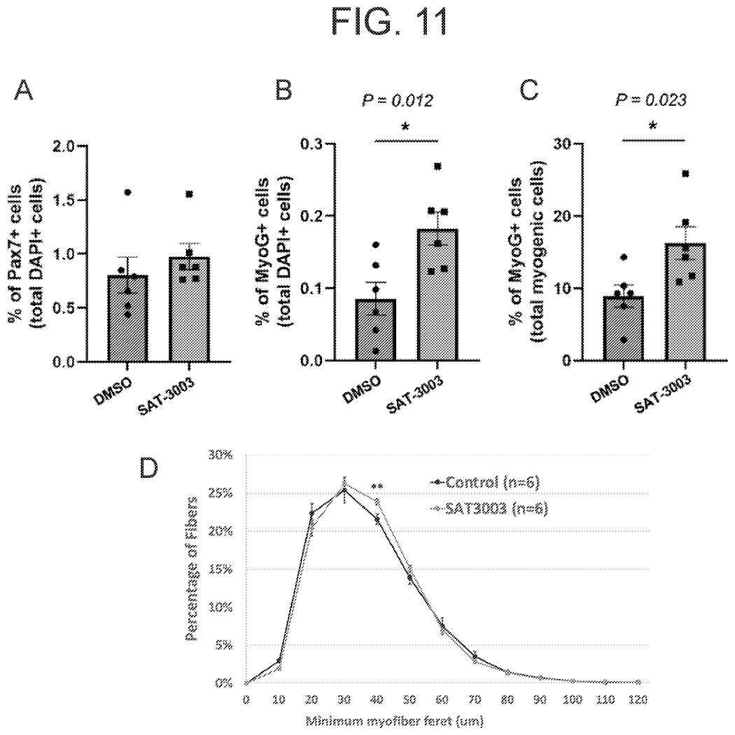

LX9211 (SAT-3003) treatment increased the prevalence of MyoG-positive (MyoG+) myogenic progenitors, as evidenced by a significant (˜100%) increase in the percentage of MyoG+ cells (FIGS. 11B and 11C) but not the % of Pax7-positive (Pax7+) cells (FIG. 11A). The LX9211 (SAT3003) treated muscles also had significant shift in distribution of minimum myofiber Feret compared to vehicle treated controls (FIG. 11D).

These data are consistent with LX9211-mediated inhibition of AAK1 inducing an increase in asymmetric muscle stem cell division in regenerating mdx muscles and stimulating myofiber regeneration. As such, these data support the use of AAK1 inhibition (e.g., using the LX9211 inhibitor) to treat diseases and injuries that would benefit from increased asymmetric satellite cell division and tissue regeneration.

### Example 4. Differential Effects of AAK1 Inhibitor Dosage Regimens on Myogenic Progenitor Density and Myofiber Feret

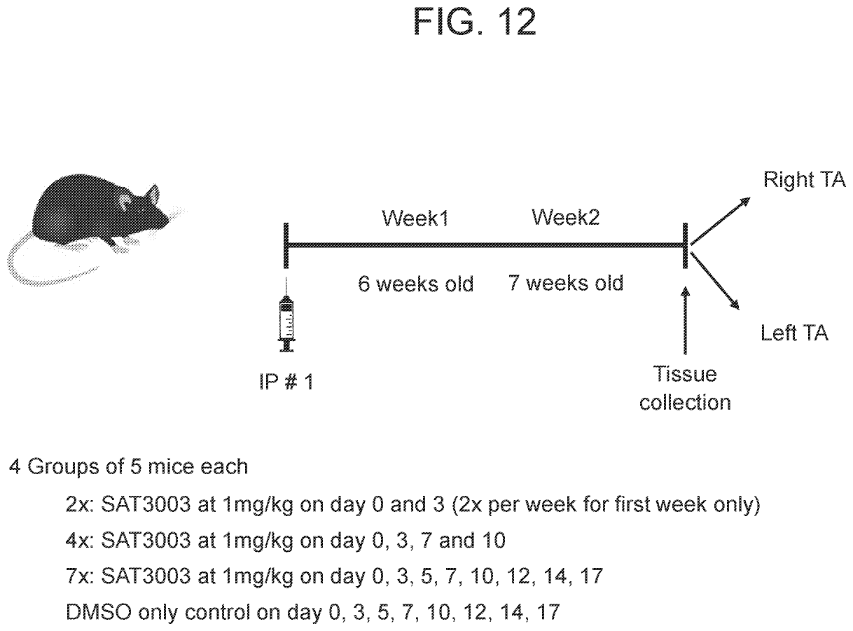

The effect of LX9211 (SAT3003)-mediated AAK1 inhibition according to different dosage regimens was next examined, using a C57BL/10ScSn-Dmd^{mdx}/J (mdx) mouse model of Duchenne muscular dystrophy. LX9211 (1 mg/kg) or DMSO (control) was administered (intraperitoneally, 10 μl/g body weight) according to the following dosage regimens: (1) day 0 and 3 (“2×”, twice a week for one week); (2) day 0, 3, 7 and 10 (“4×”, twice a week for two weeks); (3) day 0, 3, 5, 7, 10, 12, 14, 17 (“7×”, four times a week for two weeks); or (4) DMSO only (control) day 0, 3, 5, 7, 10, 12, 14, 17 (four times a week for two weeks). Ten days after CDX injection, the right TA was harvested for a single cell assay: it was processed by enzymatic digestion for mononuclear cell isolation, then filtered, plated and stained for Pax7 and MyoG prior to imaging. The left TA was harvested for histology: cryosectioned, then immunostained for Pax7 and MyoG, prior to imaging with Zeiss software (FIG. 12).

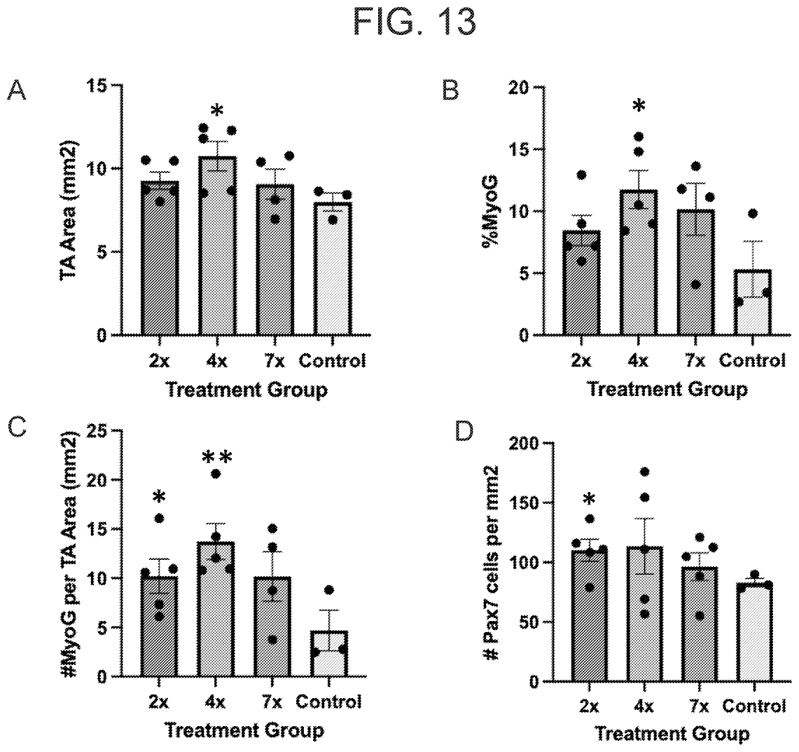

LX9211 (SAT-3003) treatment according to dosage regimen 2 (“4×”) increased muscle mass compared to vehicle treated controls (FIG. 13A). LX9211 treatment according to dosage regimen 2 also increased the prevalence of MyoG positive myogenic progenitors, as evidenced by a significant (˜100%) increase in the percentage and number of MyoG+ cells (FIGS. 13B and 13C) but not Pax7+ cells (FIG. 13D). Neither dosage regimen 1 (“2×”) or dosage regimen 3 (“7×”) significantly increased muscle mass or the percentage of MyoG+ cells, while dosage regimen 1 increased the number of both MyoG+ and Pax7+ cells.

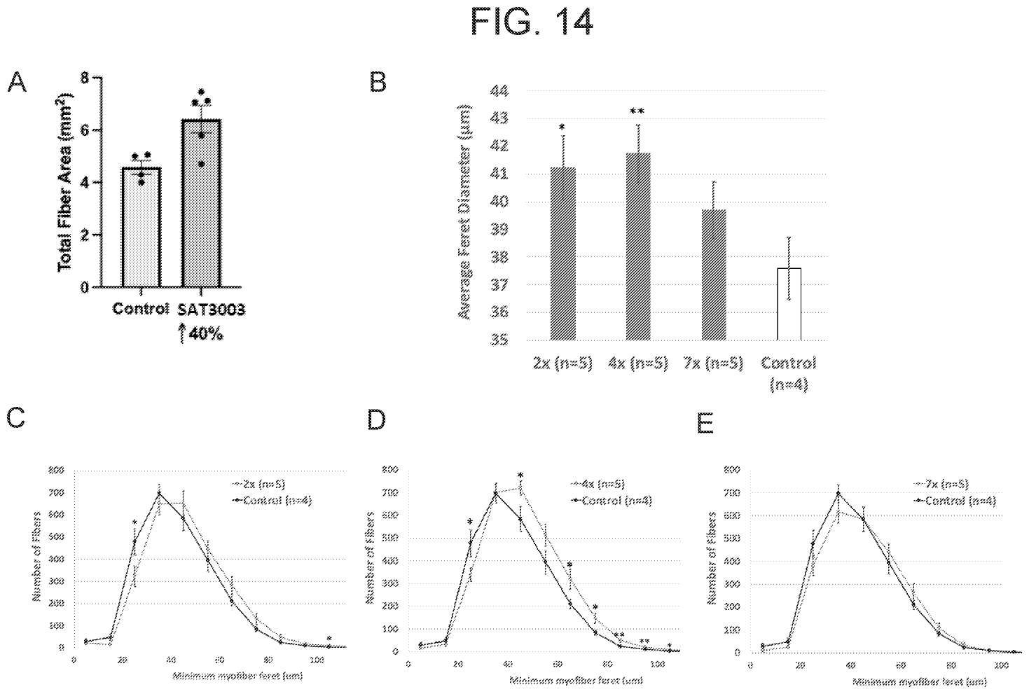

LX9211 (SAT3003) treatment according to dosage regimen 2 (“4×”) increased total TA muscle fiber area (FIG. 14A) and average Feret diameter (FIG. 14B) compared to vehicle treated controls. The animals treated with LX9211 according to dosage regimen 2 (“4×”) also had a significant shift in the distribution of minimum myofiber Feret (FIG. 14D) compared to vehicle treated controls. Neither dosage regimen 1 or 3 significantly increased the total TA muscle fiber area (FIG. 14A) or average Feret diameter (FIG. 14B), nor did they significantly shift the distribution of minimum myofiber ferret (FIGS. 14C and 14E).

These data add further support for the use of AAK1 inhibition (e.g., using LX9211) in inducing an increase in asymmetric muscle stem cell division in regenerating mdx muscles and stimulating myofiber regeneration. Moreover, these data introduce an unexpected effect of different dosage regimens in stimulating the effect of LX9211.

### Example 5. Increased Muscle Strength with Increasing Dose of AAK1 Inhibitor

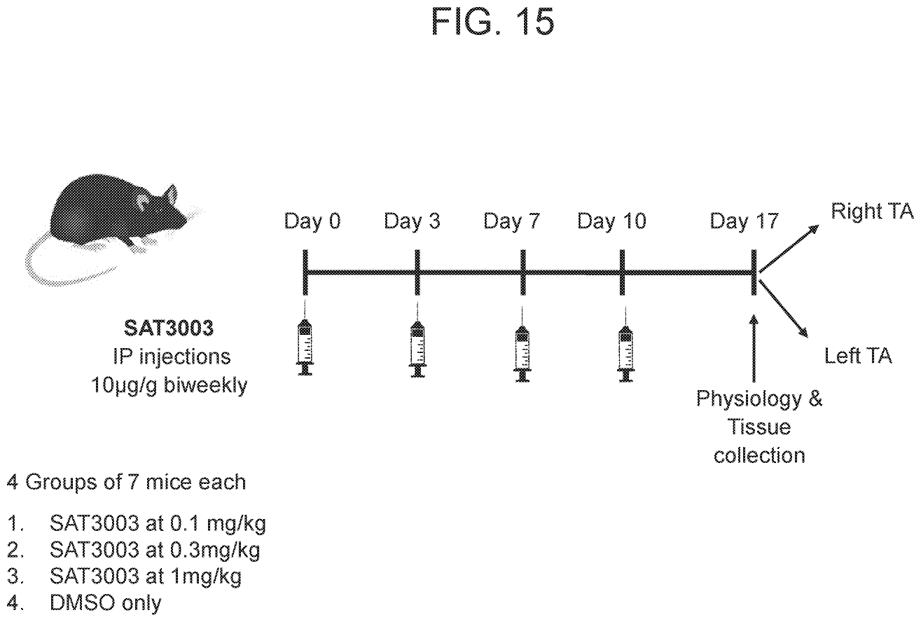

The effect of LX9211 (SAT3003)-mediated AAK1 inhibition according to different dose amounts was next examined, using a C57BL/10ScSn-Dmd^{mdx}/J (mdx) mouse model of Duchenne muscular dystrophy. LX9211 (0, 0.1, 0.3, or 1 mg/kg) was administered (intraperitoneally, 10 μl/g body weight) on days 0, 3, 7, and 10. On day 17, the right TA was subjected to physiological testing using a Dynamic Muscle Control (DMC) 300C-LR-FP muscle lever (Aurora Scientific) with ASI 610A Dynamic Muscle Control v5.500 software, and with a High-Power, Bi-Phase Stimulator and a Dual-Mode Lever System for each muscle lever. Data was analyzed using ASI 611A Dynamic Muscle Analysis v5.300 software. Briefly, (1) the mice were anesthetized; (2) dissection was performed to expose the TA tendon, attach strings to the tendon, expose the TA and the kneecap, (3) measurements were conducted using the manual trigger every 100 seconds for 15 minutes and tension at 20 mN, and (4) data was analyzed using DMCv5.500. The left TA was harvested for histology: cryosectioned, then immunostained for Pax7 and MyoG and/or laminin/WGA prior to imaging with Zeiss software (FIG. 15).

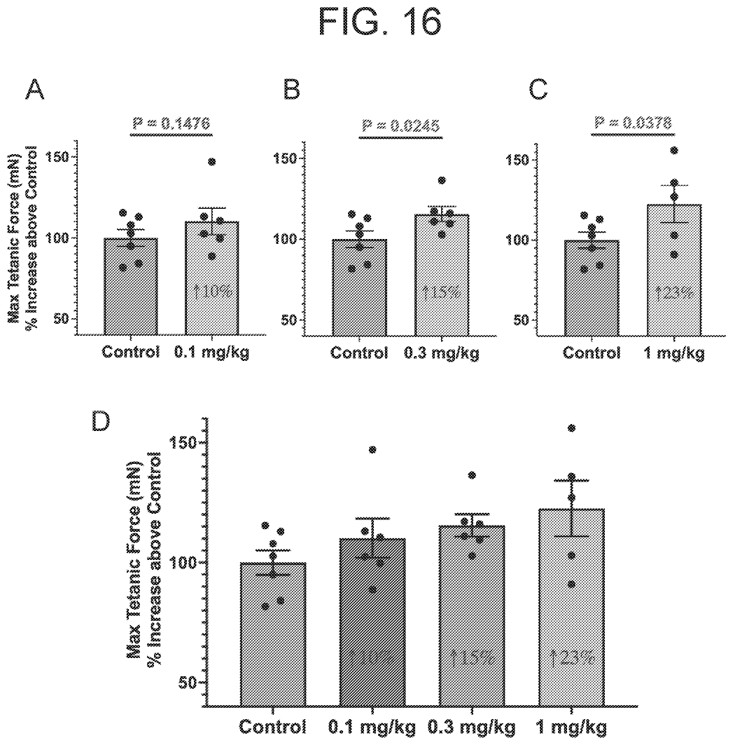

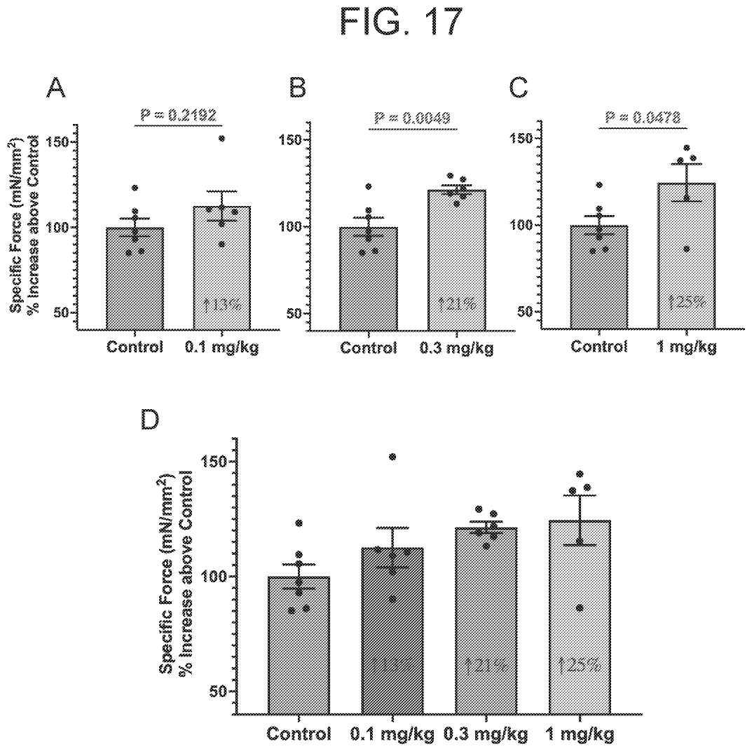

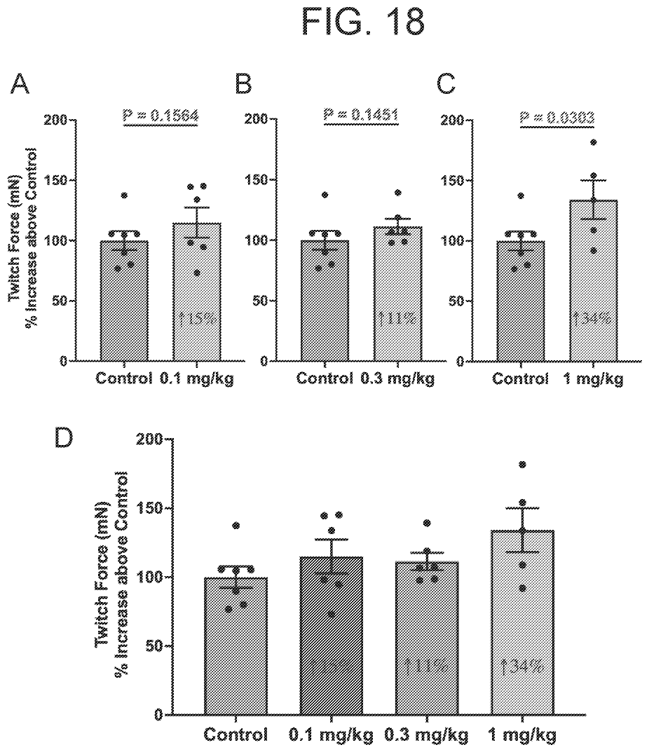

LX9211 (SAT-3003) treatment resulted in a significant increase in the Max Tetanic Force (nM, FIG. 16), Specific Force (mN/mm2, FIG. 17), and Twitch Force (mN, FIG. 18) normalized to controls. A dose-dependent effect was observed; 1 mg/kg of LX9211 SAT-3003 was associated with a larger increase in Max Tetanic Force, Specific Force, and Twitch Force, compared with lower doses.

These data add support for the use of AAK1 inhibition (e.g., using LX9211) in promoting muscle cell/tissue regeneration for treatment of muscle tissue injuries and muscle diseases, including muscular dystrophies. Moreover, these data introduce a dose-dependent effect of the AAK1 inhibitor LX9211 on muscle strength.

The various embodiments described herein can be combined to provide further embodiments.

Aspects of the embodiments can be modified, if necessary to employ concepts of the various patents, application, and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

All of the U.S. patents, U.S. patent application publications, U.S. patent applications, foreign patents, foreign patent applications and non-patent publications referred to in this specification are incorporated herein by reference, in their entireties.

1. A method for increasing asymmetric cell division of skeletal muscle stem cells, the method comprising contacting the skeletal muscle stem cells with an inhibitor of any of adaptor-associated kinase 1 (AAK1), cyclin G-associated kinase (GAK), or myristoylated and/or palmitoylated serine/threonine kinase 1 (MPSK1).

2. The method of claim 1, wherein the skeletal muscle stem cells are damaged or injured skeletal muscle stem cells or are present within damaged or injured skeletal muscle tissue.

3. The method of claim 2, wherein the muscle tissue is damaged or injured as a result of: physical injury or accident, disease, gene mutation, infection, over-use, loss of blood circulation, muscle atrophy, muscle wasting, dystrophic muscle, or ageing.

4. The method of claim 3, wherein the skeletal muscle stem cells are diseased skeletal muscle stem cells comprising a mutation associated with a muscular dystrophy, optionally Duchenne muscular dystrophy (DMD), Becker muscular dystrophy (BMD), Emery-Dreifuss muscular dystrophy, Landouzy-Dejerine muscular dystrophy, facioscapulohumeral muscular dystrophy (FSH), Limb-Girdle muscular dystrophies, von Graefe-Fuchs muscular dystrophy, oculopharyngeal muscular dystrophy (OPMD), Myotonic dystrophy (Steinert's disease) or a congenital muscular dystrophy.

5. The method of claim 3, wherein the damaged or injured muscle stem cells comprise a mutation of a dystrophin gene.

6. The method of claim 2, wherein the skeletal muscle stem cells are present within injured muscle tissue.

7. The method of any one of claims 2-6, wherein the skeletal muscle stem cells have reduced asymmetric cell division as compared to normal, healthy skeletal muscle stem cells.

8. The method of any one of claims 1-7, wherein the inhibitor inhibits expression of AAK1, GAK, or MPSK1, optionally by inhibiting transcription, translation, post-translational modification, or stability of the protein component, or the gene encoding the protein component.

9. The method of claim 8, wherein the inhibitor binds to a polynucleotide sequence that regulates expression of AAK1, GAK, or MPSK1, optionally wherein the nucleotide sequence is present within the AAK1, GAK, or MPSK1 gene.

10. The method of claim 8 or claim 9, wherein the inhibitor binds to a polynucleotide sequence that encodes AAK1, GAK, or MPSK1, or a polynucleotide sequence complementary to the polynucleotide sequence that encodes AAK1, GAK, or MPSK1, optionally wherein the polynucleotide sequence is present within the AAK1, GAK, or MPSKI gene or mRNA.

11. The method of claim 1 or claim 10, wherein the polynucleotide sequence is DNA or RNA.

12. The method of any one of claims 9-11, wherein the inhibitor comprises a polynucleotide sequence.

13. The method of claim 12, wherein the inhibitor comprises a DNA polynucleotide sequence and/or an RNA polynucleotide sequence.

14. The method of claim 12 or claim 13, wherein the inhibitor comprises a shRNA, a microRNA, a gRNA, an siRNA, an aptamer, or an antisense oligonucleotide.

15. The method of claim 14, wherein the inhibitor comprises a guide RNA targeting the AAK1 gene and a polynucleotide sequence encoding a CRISPR-Cas protein.

16. The method of any one of claims 1-7, wherein the inhibitor inhibits an activity of AAK1, GAK, or MPSK1.

17. The method of claim 16, wherein the inhibitor binds to AAK1, GAK, or MPSK1.

18. The method of claim 16 or claim 17, wherein the inhibitor comprises a polypeptide.

19. The method of claim 18, wherein the inhibitor comprises an antibody, or a functional fragment thereof, that binds to AAK1, GAK, or MPSK1.

20. The method of claim 16 or claim 17, wherein the inhibitor is an organic molecule, e.g., a small organic molecule.

21. The method of claim 20, wherein the inhibitor is selected from the group consisting of: SGC-AAK1-1, LP-935509, LP-922761, BMT-090605, BMT-124110, LP-927443, and BMS-901715.

22. The method of any one of claims 16-21, wherein the inhibitor inhibits AAK1, GAK, or MPSK1 kinase activity or AAK1, GAK, or MPSK1 ATP binding activity.

23. The method of any one of claims 1-22, wherein the inhibitor of AAKI, GAK, or MPSK1 does not substantially inhibit proliferation or cell cycle progression of the skeletal muscle stem cells.

24. The method of any one of claims 1-23, wherein the contacting occurs in vitro, in vivo, ex vivo, or in situ.

25. The method of any one of claims 1-24, wherein the cells are mammalian, optionally human.

26. The method of any one of claims 1-25, wherein the inhibitor inhibits AAK1.

27. The method of any one of claims 1-25, wherein the inhibitor inhibits GAK.

28. The method of any one of claims 1-25, wherein the inhibitor inhibits MPSK1.

29. The method of any one of claims 1-28, wherein the inhibitor is administered once every 1, 2, 3, 4, 5, 6, or 7 days.

30. The method of any one of claims 1-29, wherein the inhibitor is administered once about every 3 days.

31. The method of any one of claims 1-28, wherein the inhibitor is administered 1, 2, 3, 4, 5, 6, or 7 times per week.

32. The method of claim 31, wherein the inhibitor is administered 2 times per week.

33. The method of any one of claims 1-32, wherein the inhibitor is administered at a dose of about 0.01 mg/kg to about 300 mg/kg.

34. The method of any one of claims 1-33, wherein the inhibitor is administered at a dose of about 0.1 mg/kg to about 20 mg/kg.

35. The method of any one of claims 1-34, wherein the inhibitor is administered at a dose of about 0.1, about 0.3, about 0.7, about 1, about 2, about 3, about 4, about 5, about 6, about 7, about 8, about 9, about 10, about 11, about 12, about 13, about 14, about 15, about 16, about 17, about 18, about 19 or about 20 mg/kg.

36. The method of any one of claims 1-35, wherein the inhibitor is administered at a dose of about 1 mg/kg.

37. A method for increasing skeletal muscle tissue growth or regeneration in a subject, comprising administering to the subject an inhibitor of AAK1, GAK, or MPSK1.

38. The method of claim 37, wherein the subject has damaged or injured skeletal muscle tissue.

39. The method of claim 38, wherein the skeletal muscle tissue is damaged or injured as a result of: physical injury or accident, disease, gene mutation, infection, over-use, loss of blood circulation, muscle atrophy, muscle wasting, dystrophic muscle, or ageing.

40. The method of claim 39, wherein the subject has a muscular dystrophy, optionally Duchenne muscular dystrophy (DMD), Becker muscular dystrophy (BMD), Emery-Dreifuss muscular dystrophy, Landouzy-Dejerine muscular dystrophy, facioscapulohumeral muscular dystrophy (FSH), Limb-Girdle muscular dystrophies, von Graefe-Fuchs muscular dystrophy, oculopharyngeal muscular dystrophy (OPMD), Myotonic dystrophy (Steinert's disease) or a congenital muscular dystrophy.

41. The method of claim any one of claims 37-40, wherein the subject comprises a mutation of a dystrophin gene.

42. The method of any one of claims 37-41, wherein skeletal muscle stem cells within the skeletal muscle tissue have reduced asymmetric cell division as compared to normal, healthy skeletal muscle stem cells.

43. The method of any one of claims 37-42, wherein the inhibitor of AAK1, GAK, or MPSK1 does not substantially inhibit proliferation or cell cycle progression of the subject's skeletal muscle stem cells.

44. The method of any one of claims 37-43, wherein the method increases skeletal muscle tissue regeneration in the subject.

45. The method of any one of claims 37-44, wherein the subject is a mammal, optionally a human.

46. The method of any one of claims 37-45, wherein the inhibitor of AAK1, GAK, or MPSK1 is administered to the subject systemically or locally, optionally at a site of tissue damage or injury.

47. The method of any one of claims 37-46, wherein the inhibitor inhibits expression of AAK1, GAK, or MPSK1, optionally by inhibiting transcription, translation, post-translational modification, or stability of the protein component, or the gene encoding the protein component.

48. The method of claim 47, wherein the inhibitor binds to a polynucleotide sequence that regulates expression of AAK1, GAK, or MPSK1, optionally wherein the nucleotide sequence is present within the AAK1, GAK, or MPSK1 gene.

49. The method of claim 47 or claim 48, wherein the inhibitor binds to a polynucleotide sequence that encodes AAK1, GAK, or MPSK1, or a polynucleotide sequence complementary to the polynucleotide sequence that encodes AAK1, GAK, or MPSK1, optionally wherein the polynucleotide sequence is present within the AAK1, GAK, or MPSK1 gene or mRNA.

50. The method of any claim 48 or claim 49, wherein the polynucleotide sequence is DNA or RNA.

51. The method of any one of claims 47-50, wherein the inhibitor comprises a polynucleotide sequence.

52. The method of claim 51, wherein the inhibitor comprises a DNA polynucleotide sequence and/or an RNA polynucleotide sequence.

53. The method of claim 51 or claim 52, wherein the inhibitor comprises a shRNA, a microRNA, a gRNA, an siRNA, an aptamer, or an antisense oligonucleotide.

54. The method of claim 53, wherein the inhibitor comprises a guide RNA targeting the AAK1 gene and a polynucleotide sequence encoding a CRISPR-Cas protein.

55. The method of any one of claims 37-46, wherein the inhibitor inhibits an activity of AAK1, GAK, or MPSK1.

56. The method of claim 55, wherein the inhibitor binds to AAK1, GAK, or MPSK1.

57. The method of claim 55 or claim 56, wherein the inhibitor comprises a polypeptide.

58. The method of claim 57, wherein the inhibitor comprises an antibody, or a functional fragment thereof, that binds to AAK1, GAK, or MPSK1.

56. The method of claim 55 or claim 56, wherein the inhibitor is an organic molecule, e.g., a small organic molecule.

60. The method of claim 59, wherein the inhibitor is selected from the group consisting of: SGC-AAK1-1, LP-935509, LP-922761, BMT-090605, BMT-124110, LP-927443, and BMS-901715.

61. The method of any one of claims 55-60, wherein the inhibitor inhibits AAK1, GAK, or MPSK1 kinase activity or AAK1, GAK, or MPSK1 ATP binding activity.

62. The method of any one of claims 37-61, wherein the inhibitor inhibits AAK1.

63. The method of any one of claims 37-61, wherein the inhibitor inhibits GAK.

64. The method of any one of claims 37-61, wherein the inhibitor inhibits MPSK1.

65. The method of any one of claims 37-64, wherein the inhibitor is administered once every 1, 2, 3, 4, 5, 6, or 7 days.

66. The method of any one of claims 37-65, wherein the inhibitor is administered once about every 3 days.

67. The method of any one of claims 37-64, wherein the inhibitor is administered 1, 2, 3, 4, 5, 6, or 7 times per week.

68. The method of claim 67, wherein the inhibitor is administered 2 times per week.

69. The method of any one of claims 37-68, wherein the inhibitor is administered at a dose of about 0.01 mg/kg to about 300 mg/kg.

70. The method of any one of claims 37-69, wherein the inhibitor is administered at a dose of about 0.1 mg/kg to about 20 mg/kg.

71. The method of any one of claims 37-70, wherein the inhibitor is administered at a dose of about 0.1, about 0.3, about 0.7, about 1, about 2, about 3, about 4, about 5, about 6, about 7, about 8, about 9, about 10, about 11, about 12, about 13, about 14, about 15, about 16, about 17, about 18, about 19 or about 20 mg/kg.

72. The method of any one of claims 37-71, wherein the inhibitor is administered at a dose of about 1 mg/kg.

73. A method for treating a muscular dystrophy, comprising administering to a subject in need thereof an inhibitor of AAK1, GAK, or MPSK1.

74. The method of claim 73, wherein the subject has a muscular dystrophy selected from the group consisting of: Duchenne muscular dystrophy (DMD), Becker muscular dystrophy (BMD), Emery-Dreifuss muscular dystrophy, Landouzy-Dejerine muscular dystrophy, facioscapulohumeral muscular dystrophy (FSH), Limb-Girdle muscular dystrophies, von Graefe-Fuchs muscular dystrophy, oculopharyngeal muscular dystrophy (OPMD), Myotonic dystrophy (Steinert's disease) and a congenital muscular dystrophy.

75. The method of claim 73 or claim 74, wherein the subject comprises a mutation of a dystrophin gene.

76. The method of any one of claims 73-75, wherein skeletal muscle stem cells within the subject have reduced asymmetric cell division as compared to normal, healthy skeletal muscle stem cells.

77. The method of any one of claims 73-76, wherein the inhibitor of AAK1, GAK, or MPSK1 does not substantially inhibit proliferation or cell cycle progression of the subject's skeletal muscle stem cells.

78. The method of any one of claims 73-77, wherein the method increases skeletal muscle tissue regeneration in the subject.

79. The method of any one of claims 73-78, wherein the subject is a mammal, optionally a human.

80. The method of any one of claims 73-79, wherein the inhibitor of AAK1, GAK, or MPSK1 is administered to the subject systemically or locally, optionally at a site of tissue damage or injury.

81. The method of any one of claims 73-80, wherein the inhibitor inhibits expression of AAK1, GAK, or MPSK1, optionally by inhibiting transcription, translation, post-translational modification, or stability of the protein component, or the gene encoding the protein component.

82. The method of claim 81, wherein the inhibitor binds to a polynucleotide sequence that regulates expression of AAK1, GAK, or MPSK1, optionally wherein the nucleotide sequence is present within the AAK1, GAK, or MPSK1 gene.

83. The method of claim 81 or claim 82, wherein the inhibitor binds to a polynucleotide sequence that encodes AAK1, GAK, or MPSK1, or a polynucleotide sequence complementary to the polynucleotide sequence that encodes AAK1, GAK, or MPSK1, optionally wherein the polynucleotide sequence is present within the AAK1, GAK, or MPSK1 gene or mRNA.

84. The method of any one of claim 82 or claim 83, wherein the polynucleotide sequence is DNA or RNA.

85. The method of any one of claims 81-84, wherein the inhibitor comprises a polynucleotide sequence.

86. The method of claim 85, wherein the inhibitor comprises a DNA polynucleotide sequence and/or an RNA polynucleotide sequence.

87. The method of claim 85 or claim 86, wherein the inhibitor comprises a shRNA, a microRNA, a gRNA, an siRNA, an aptamer, or an antisense oligonucleotide.

88. The method of claim 87, wherein the inhibitor comprises a guide RNA targeting the AAK1 gene and a polynucleotide sequence encoding a CRISPR-Cas protein.

89. The method of any one of claims 73-80, wherein the inhibitor inhibits an activity of AAK1, GAK, or MPSK1.

90. The method of claim 89, wherein the inhibitor binds to AAK1, GAK, or MPSK1.

91. The method of claim 89 or claim 90, wherein the inhibitor comprises a polypeptide.

92. The method of claim 91, wherein the inhibitor comprises an antibody, or a functional fragment thereof, that binds to AAK1, GAK, or MPSK1.

93. The method of claim 89 or claim 90, wherein the inhibitor is an organic molecule, e.g., a small organic molecule.

94. The method of claim 93, wherein the inhibitor is selected from the group consisting of: SGC-AAK1-1, LP-935509, LP-922761, BMT-090605, BMT-124110, LP-927443, and BMS-901715.

95. The method of any one of claims 89-94, wherein the inhibitor inhibits AAK1, GAK, or MPSK1 kinase activity or AAK1, GAK, or MPSK1 ATP binding activity.

96. The method of any one of claims 73-95, wherein the inhibitor inhibits AAK1.

97. The method of any one of claims 73-95, wherein the inhibitor inhibits GAK. 98 The method of any one of claims 73-95, wherein the inhibitor inhibits MPSK1. 99 The method of any one of claims 73-98, wherein the inhibitor is administered once every 1, 2, 3, 4, 5, 6, or 7 days.

100. The method of any one of claims 73-99, wherein the inhibitor is administered once about every 3 days.

101. The method of any one of claims 73-98, wherein the inhibitor is administered 1, 2, 3, 4, 5, 6, or 7 times per week.

102. The method of claim 101, wherein the inhibitor is administered 2 times per week.

103. The method of any one of claims 73-102, wherein the inhibitor is administered at a dose of about 0.01 mg/kg to about 300 mg/kg.

104. The method of any one of claims 73-103, wherein the inhibitor is administered at a dose of about 0.1 mg/kg to about 20 mg/kg.

105. The method of any one of claims 73-104, wherein the inhibitor is administered at a dose of about 0.1, about 0.3, about 0.7, about 1, about 2, about 3, about 4, about 5, about 6, about 7, about 8, about 9, about 10, about 11, about 12, about 13, about 14, about 15, about 16, about 17, about 18, about 19 or about 20 mg/kg.

106. The method of any one of claims 73-105, wherein the inhibitor is administered at a dose of about 1 mg/kg.


SEQUENCE LISTING (24 sequences)

SEQ ID NO: 1 - protein, 961 aa, Homo sapiens
MKKFFDSRREQGGSGLGSGSSGGGGSTSGLGSGYIGRVFGIGRQQVTVDEVLAEGGFAIVFLVRTSNGMKCALKRMFVNNEHDLQVCKREIQIMRDLSGHKNIVGYIDSSINNVSSGDVWEVLILMDFCRGGQVVNLMNQRLQTGFTENEVLQIFCDTCEAVARLHQCKTPIIHRDLKVENILLHDRGHYVLCDFGSATNKFQNPQTEGVNAVEDEIKKYTTLSYRAPEMVNLYSGKIITTKADIWALGCLLYKLCYFTLPFGESQVAICDGNFTIPDNSRYSQDMHCLIRYMLEPDPDKRPDIYQVSYFSFKLLKKECPIPNVQNSPIPAKLPEPVKASEAAAKKTQPKARLTDPIPTTETSIAPRQRPKAGQTQPNPGILPIQPALTPRKRATVQPPPQAAGSSNQPGLLASVPQPKPQAPPSQPLPQTQAKQPQAPPTPQQTPSTQAQGLPAQAQATPQHQQQLFLKQQQQQQQPPPAQQQPAGTFYQQQQAQTQQFQAVHPATQKPAIAQFPVVSQGGSQQQLMQNFYQQQQQQQQQQQQQQLATALHQQQLMTQQAALQQKPTMAAGQQPQPQPAAAPQPAPAQEPAIQAPVRQQPKVQTTPPPAVQGQKVGSLTPPSSPKTQRAGHRRILSDVTHSAVFGVPASKSTQLLQAAAAEASLNKSKSATTTPSGSPRTSQQNVYNPSEGSTWNPFDDDNFSKLTAEELLNKDFAKLGEGKHPEKLGGSAESLIPGFQSTQGDAFATTSFSAGTAEKRKGGQTVDSGLPLLSVSDPFIPLQVPDAPEKLIEGLKSPDTSLLLPDLLPMTDPFGSTSDAVIEKADVAVESLIPGLEPPVPQRLPSQTESVTSNRTDSLTGEDSLLDCSLLSNPTTDLLEEFAPTAISAPVHKAAEDSNLISGFDVPEGSDKVAEDEFDPIPVLITKNPQGGHSRNSSGSSESSLPNLARSLLLVDQLIDL

SEQ ID NO: 2 - protein, 961 aa, Homo sapiens; identical to SEQ ID NO: 1

SEQ ID NO: 3 - protein, 863 aa, Homo sapiens
MKKFFDSRREQGGSGLGSGSSGGGGSTSGLGSGYIGRVFGIGRQQVTVDEVLAEGGFAIVFLVRTSNGMKCALKRMFVNNEHDLQVCKREIQIMRDLSGHKNIVGYIDSSINNVSSGDVWEVLILMDFCRGGQVVNLMNQRLQTGFTENEVLQIFCDTCEAVARLHQCKTPIIHRDLKVENILLHDRGHYVLCDFGSATNKFQNPQTEGVNAVEDEIKKYTTLSYRAPEMVNLYSGKIITTKADIWALGCLLYKLCYFTLPFGESQVAICDGNFTIPDNSRYSQDMHCLIRYMLEPDPDKRPDIYQVSYFSFKLLKKECPIPNVQNSPIPAKLPEPVKASEAAAKKTQPKARLTDPIPTTETSIAPRQRPKAGQTQPNPGILPIQPALTPRKRATVQPPPQAAGSSNQPGLLASVPQPKPQAPPSQPLPQTQAKQPQAPPTPQQTPSTQAQGLPAQAQATPQHQQQLFLKQQQQQQQPPPAQQQPAGTFYQQQQAQTQQFQAVHPATQKPAIAQFPVVSQGGSQQQLMQNFYQQQQQQQQQQQQQQLATALHQQQLMTQQAALQQKPTMAAGQQPQPQPAAAPQPAPAQEPAIQAPVRQQPKVQTTPPPAVQGQKVGSLTPPSSPKTQRAGHRRILSDVTHSAVFGVPASKSTQLLQAAAAEASLNKSKSATTTPSGSPRTSQQNVYNPSEGSTWNPFDDDNFSKLTAEELLNKDFAKLGEGKHPEKLGGSAESLIPGFQSTQGDAFATTSFSAGTAEKRKGGQTVDSGLPLLSVSDPFIPLQVPDAPEKLIEGLKSPDTSLLLPDLLPMTDPFGSTSDAVIGKVIISVSSVMHDMCACFKNDKYLVNQSLGNSPATPEAKAI

SEQ ID NO: 4 - protein, 1161 aa, Homo sapiens
MKKFSRMPKSEGGSGGGAAGGGAGGAGAGAGCGSGGSSVGVRVFAVGRHQVTLEESLAEGGFSTVFLVRTHGGIRCALKRMYVNNMPDLNVCKREITIMKELSGHKNIVGYLDCAVNSISDNVWEVLILMEYCRAGQVVNQMNKKLQTGFTEPEVLQIFCDTCEAVARLHQCKTPIIHRDLKVENILLNDGGNYVLCDFGSATNKFLNPQKDGVNVVEEEIKKYTTLSYRAPEMINLYGGKPITTKADIWALGCLLYKLCFFTLPFGESQVAICDGNFTIPDNSRYSRNIHCLIRFMLEPDPEHRPDIFQVSYFAFKFAKKDCPVSNINNSSIPSALPEPMTASEAAARKSQIKARITDTIGPTETSIAPRQRPKANSATTATPSVLTIQSSATPVKVLAPGEFGNHRPKGALRPGNGPEILLGQGPPQQPPQQHRVLQQLQQGDWRLQQLHLQHRHPHQQQQQQQQQQQQQQQQQQQQQQQQQQQHHHHHHHHLLQDAYMQQYQHATQQQQMLQQQFLMHSVYQPQPSASQYPTMMPQYQQAFFQQQMLAQHQPSQQQASPEYLTSPQEFSPALVSYTSSLPAQVGTIMDSSYSANRSVADKEAIANFTNQKNISNPPDMSGWNPFGEDNFSKLTEEELLDREFDLLRSNRLEERASSDKNVDSLSAPHNHPPEDPFGSVPFISHSGSPEKKAEHSSINQENGTANPIKNGKTSPASKDQRTGKKTSVQGQVQKGNDESESDFESDPPSPKSSEEEEQDDEEVLQGEQGDFNDDDTEPENLGHRPLLMDSEDEEEEEKHSSDSDYEQAKAKYSDMSSVYRDRSGSGPTQDLNTILLTSAQLSSDVAVETPKQEFDVFGAVPFFAVRAQQPQQEKNEKNLPQHRFPAAGLEQEEFDVFTKAPFSKKVNVQECHAVGPEAHTIPGYPKSVDVFGSTPFQPFLTSTSKSESNEDLFGLVPFDEITGSQQQKVKQRSLQKLSSRQRRTKQDMSKSNGKRHHGTPTSTKKTLKPTYRTPERARRHKKVGRRDSQSSNEFLTISDSKENISVALTDGKDRGNVLQPEESLLDPFGAKPFHSPDLSWHPPHQGLSDIRADHNTVLPGRPRQNSLHGSFHSADVLKMDDFGAVPFTELVVQSITPHQSQQSQPVELDPFGAAPFPSKQ

SEQ ID NO: 5 - protein, 1311 aa, Homo sapiens
MSLLQSALDFLAGPGSLGGASGRDQSDFVGQTVELGELRLRVRRVLAEGGFAFVYEAQDVGSGREYALKRLLSNEEEKNRAIIQEVCFMKKLSGHPNIVQFCSAASIGKEESDTGQAEFLLLTELCKGQLVEFLKKMESRGPLSCDTVLKIFYQTCRAVQHMHRQKPPIIHRDLKVENLLLSNQGTIKLCDFGSATTISHYPDYSWSAQRRALVEEEITRNTTPMYRTPEIIDLYSNFPIGEKQDIWALGCILYLLCFRQHPFEDGAKLRIVNGKYSIPPHDTQYTVFHSLIRAMLQVNPEERLSIAEVVHQLQEIAAARNVNPKSPITELLEQNGGYGSATLSRGPPPPVGPAGSGYSGGLALAEYDQPYGGFLDILRGGTERLFTNLKDTSSKVIQSVANYAKGDLDISYITSRIAVMSFPAEGVESALKNNIEDVRLFLDSKHPGHYAVYNLSPRTYRPSRFHNRVSECGWAARRAPHLHTLYNICRNMHAWLRQDHKNVCVVHCMDGRAASAVAVCSFLCFCRLFSTAEAAVYMFSMKRCPPGIWPSHKRYIEYMCDMVAEEPITPHSKPILVRAVVMTPVPLFSKQRSGCRPFCEVYVGDERVASTSQEYDKMRDFKIEDGKAVIPLGVTVQGDVLIVIYHARSTLGGRLQAKMASMKMFQIQFHTGFVPRNATTVKFAKYDLDACDIQEKYPDLFQVNLEVEVEPRDRPSREAPPWENSSMRGLNPKILFSSREEQQDILSKFGKPELPRQPGSTAQYDAGAGSPEAEPTDSDSPPSSSADASRFLHTLDWQEEKEAETGAENASSKESESALMEDRDESEVSDEGGSPISSEGQEPRADPEPPGLAAGLVQQDLVFEVETPAVLPEPVPQEDGVDLLGLHSEVGAGPAVPPQACKAPSSNTDLLSCLLGPPEAASQGPPEDLLSEDPLLLASPAPPLSVQSTPRGGPPAAADPFGPLLPSSGNNSQPCSNPDLFGEFLNSDSVTVPPSFPSAHSAPPPSCSADFLHLGDLPGEPSKMTASSSNPDLLGGWAAWTETAASAVAPTPATEGPLFSPGGQPAPCGSQASWTKSQNPDPFADLGDLSSGLQGSPAGFPPGGFIPKTATTPKGSSSWQTSRPPAQGASWPPQAKPPPKACTQPRPNYASNFSVIGAREERGVRAPSFAQKPKVSENDFEDLLSNQGFSSRSDKKGPKTIAEMRKQDLAKDTDPLKLKLLDWIEGKERNIRALLSTLHTVLWDGESRWTPVGMADLVAPEQVKKHYRRAVLAVHPDKAAGQPYEQHAKMIFMELNDAWSEFENQGSRPLF

SEQ ID NO: 6 - protein, 305 aa, Homo sapiens
MGHALCVCSRGTVIIDNKRYLFIQKLGEGGFSYVDLVEGLHDGHFYALKRILCHEQQDREEAQREADMHRLFNHPNILRLVAYCLRERGAKHEAWLLLPFFKRGTLWNEIERLKDKGNFLTEDQILWLLLGICRGLEAIHAKGYAHRDLKPTNILLGDEGQPVLMDLGSMNQACIHVEGSRQALTLQDWAAQRCTISYRAPELFSVQSHCVIDERTDVWSLGCVLYAMMFGEGPYDMVFQKGDSVALAVQNQLSIPQSPRHSSALRQLLNSMMTVDPHQRPHIPLLLSQLEALQPPAPGQHTTQI

SEQ ID NO: 7 - protein, 465 aa, Homo sapiens
MAMTGSTPCSSMSNHTKERVTMTKVTLENFYSNLIAQHEEREMRQKKLEKVMEEEGLKDEEKRLRRSAHARKETEFLRLKRTRLGLEDFESLKVIGRGAFGEVRLVQKKDTGHVYAMKILRKADMLEKEQVGHIRAERDILVEADSLWVVKMFYSFQDKLNLYLIMEFLPGGDMMTLLMKKDTLTEEETQFYIAETVLAIDSIHQLGFIHRDIKPDNLLLDSKGHVKLSDFGLCTGLKKAHRTEFYRNLNHSLPSDFTFQNMNSKRKAETWKRNRRQLAFSTVGTPDYIAPEVFMQTGYNKLCDWWSLGVIMYEMLIGYPPFCSETPQETYKKVMNWKETLTFPPEVPISEKAKDLILRFCCEWEHRIGAPGVEEIKSNSFFEGVDWEHIRERPAAISIEIKSIDDTSNFDEFPESDILKPTVATSNHPETDYKNKDWVFINYTYKRFEGLTARGAIPSYMKAAK

SEQ ID NO: 8 - protein, 464 aa, Homo sapiens
MAMTAGTTTTFPMSNHTRERVTVAKLTLENFYSNLILQHEERETRQKKLEVAMEEEGLADEEKKLRRSQHARKETEFLRLKRTRLGLDDFESLKVIGRGAFGEVRLVQKKDTGHIYAMKILRKSDMLEKEQVAHIRAERDILVEADGAWVVKMFYSFQDKRNLYLIMEFLPGGDMMTLLMKKDTLTEEETQFYISETVLAIDAIHQLGFIHRDIKPDNLLLDAKGHVKLSDFGLCTGLKKAHRTEFYRNLTHNPPSDFSFQNMNSKRKAETWKKNRRQLAYSTVGTPDYIAPEVFMQTGYNKLCDWWSLGVIMYEMLIGYPPFCSETPQETYRKVMNWKETLVFPPEVPISEKAKDLILRFCIDSENRIGNSGVEEIKGHPFFEGVDWEHIRERPAAIPIEIKSIDDTSNFDDFPESDILQPVPNTTEPDYKSKDWVFLNYTYKRFEGLTQRGSIPTYMKAGKL

SEQ ID NO: 9 - RNA, 19 nt, synthetic construct
  source 1..19: mol_type = other RNA
gaaggtggattcgctcttg

SEQ ID NO: 10 - RNA, 19 nt, synthetic construct
  source 1..19: mol_type = other RNA
ggactcaaatctcctgaca

SEQ ID NO: 11 - RNA, 19 nt, synthetic construct
  source 1..19: mol_type = other RNA
gcagatatttgggctctag

SEQ ID NO: 12 - RNA, 19 nt, synthetic construct
  source 1..19: mol_type = other RNA
aaatgtgccttgaaacgta

SEQ ID NO: 13 - protein, 19 aa, synthetic construct
GCAGGTATCACCCGAGTAT

SEQ ID NO: 14 - protein, 19 aa, synthetic construct
TATCCTACTTTGCGTTTAA

SEQ ID NO: 15 - RNA, 19 nt, synthetic construct
  source 1..19: mol_type = other RNA
gggaagtgcttatcttaat

SEQ ID NO: 16 - RNA, 19 nt, synthetic construct
  source 1..19: mol_type = other RNA
gctcaagtccactatgtaa

SEQ ID NO: 17 - RNA, 19 nt, synthetic construct
  source 1..19: mol_type = other RNA
gagggaggctgcaggctaa

SEQ ID NO: 18 - RNA, 19 nt, synthetic construct
  source 1..19: mol_type = other RNA
gaccaaacagcaagactta

SEQ ID NO: 19 - RNA, 19 nt, synthetic construct
  source 1..19: mol_type = other RNA
tggcagagagtatgcatta

SEQ ID NO: 20 - RNA, 19 nt, synthetic construct
  source 1..19: mol_type = other RNA
cctggatgcttgtgatatt

SEQ ID NO: 21 - RNA, 19 nt, synthetic construct
  source 1..19: mol_type = other RNA
gaaagaacgaggtgctaag

SEQ ID NO: 22 - RNA, 19 nt, synthetic construct
  source 1..19: mol_type = other RNA
tcagtcagttggaggcatt

SEQ ID NO: 23 - RNA, 19 nt, synthetic construct
  source 1..19: mol_type = other RNA
acccaaatctgatcaaatc

SEQ ID NO: 24 - RNA, 19 nt, synthetic construct
  source 1..19: mol_type = other RNA
ggacttgggttctatgaat